SEQUENCE LISTING (159 sequences)

SEQ ID NO: 1 - protein, 508 aa, synthetic construct
  REGION 1..508: note = A025001562
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSGGGSEVQLVESGGGVVQPGGSLRLSCAASGGVFRLNSMGWFRQAPGKQRELVAIITSDGDTNYADSVKGRFTISRDQAKNTVYLQMNSLRPEDTALYYCQITYSTSSYSFPINTWGQGTLVTVSSGGGGSGGGSEVQLVESGGGVVQPGGSLRLSCAASGVIFSGNVMGWYRRQAPGKEREWVAAIAEGGSILYRDSVKGRFTISRDNAKNTVYLQMNSLRPEDTALYYCNSHPPVLPYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 2 - protein, 117 aa, synthetic construct
  REGION 1..117: note = TCR VHH
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSS

SEQ ID NO: 3 - protein, 121 aa, synthetic construct
  REGION 1..121: note = CD33 VHH
EVQLVESGGGVVQPGGSLRLSCAASGGVFRLNSMGWFRQAPGKQRELVAIITSDGDTNYADSVKGRFTISRDQAKNTVYLQMNSLRPEDTALYYCQITYSTSSYSFPINTWGQGTLVTVSS

SEQ ID NO: 4 - protein, 116 aa, synthetic construct
  REGION 1..116: note = CD123 VHH
EVQLVESGGGVVQPGGSLRLSCAASGVIFSGNVMGWYRRQAPGKEREWVAAIAEGGSILYRDSVKGRFTISRDNAKNTVYLQMNSLRPEDTALYYCNSHPPVLPYWGQGTLVTVSS

SEQ ID NO: 5 - protein, 115 aa, synthetic construct
  REGION 1..115: note = ALB23002
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 6 - protein, 10 aa, synthetic construct
  REGION 1..10: note = CDR1
GYVHKINFYG

SEQ ID NO: 7 - protein, 10 aa, synthetic construct
  REGION 1..10: note = CDR1
GGVFRLNSMG

SEQ ID NO: 8 - protein, 10 aa, synthetic construct
  REGION 1..10: note = CDR1
GVIFSGNVMG

SEQ ID NO: 9 - protein, 10 aa, synthetic construct
  REGION 1..10: note = CDR1
GFTFRSFGMS

SEQ ID NO: 10 - protein, 9 aa, synthetic construct
  REGION 1..9: note = CDR2
HISIGDQTD

SEQ ID NO: 11 - protein, 9 aa, synthetic construct
  REGION 1..9: note = CDR2
IITSDGDTN

SEQ ID NO: 12 - protein, 9 aa, synthetic construct
  REGION 1..9: note = CDR2
AIAEGGSIL

SEQ ID NO: 13 - protein, 10 aa, synthetic construct
  REGION 1..10: note = CDR2
SISGSGSDTL

SEQ ID NO: 14 - protein, 9 aa, synthetic construct
  REGION 1..9: note = CDR3
LSRIWPYDY

SEQ ID NO: 15 - protein, 13 aa, synthetic construct
  REGION 1..13: note = CDR3
TYSTSSYSFPINT

SEQ ID NO: 16 - protein, 7 aa, synthetic construct
  REGION 1..7: note = CDR3
HPPVLPY

SEQ ID NO: 17 - protein, 6 aa, synthetic construct
  REGION 1..6: note = CDR3
GGSLSR

SEQ ID NO: 18 - protein, 25 aa, synthetic construct
  REGION 1..25: note = FR1
DVQLVESGGGVVQPGGSLRLSCVAS

SEQ ID NO: 19 - protein, 25 aa, synthetic construct
  REGION 1..25: note = FR1
EVQLVESGGGVVQPGGSLRLSCAAS

SEQ ID NO: 20 - protein, 25 aa, synthetic construct
  REGION 1..25: note = FR1
EVQLVESGGGVVQPGGSLRLSCAAS

SEQ ID NO: 21 - protein, 25 aa, synthetic construct
  REGION 1..25: note = FR1
EVQLVESGGGVVQPGGSLRLSCAAS

SEQ ID NO: 22 - protein, 14 aa, synthetic construct
  REGION 1..14: note = FR2
WYRQAPGKEREKVA

SEQ ID NO: 23 - protein, 14 aa, synthetic construct
  REGION 1..14: note = FR2
WFRQAPGKQRELVA

SEQ ID NO: 24 - protein, 15 aa, synthetic construct
  REGION 1..15: note = FR2
WYRRQAPGKEREWVA

SEQ ID NO: 25 - protein, 14 aa, synthetic construct
  REGION 1..14: note = FR2
WVRQAPGKGPEWVS

SEQ ID NO: 26 - protein, 39 aa, synthetic construct
  REGION 1..39: note = FR3
YADSAKGRFTISRDESKNTVYLQMNSLRPEDTAAYYCRA

SEQ ID NO: 27 - protein, 39 aa, synthetic construct
  REGION 1..39: note = FR3
YADSVKGRFTISRDQAKNTVYLQMNSLRPEDTALYYCQI

SEQ ID NO: 28 - protein, 39 aa, synthetic construct
  REGION 1..39: note = FR3
YRDSVKGRFTISRDNAKNTVYLQMNSLRPEDTALYYCNS

SEQ ID NO: 29 - protein, 39 aa, synthetic construct
  REGION 1..39: note = FR3
YADSVKGRFTISRDNSKNTLYLQMNSLRPEDTALYYCTI

SEQ ID NO: 30 - protein, 11 aa, synthetic construct
  REGION 1..11: note = FR4
WGQGTLVTVSS

SEQ ID NO: 31 - protein, 11 aa, synthetic construct
  REGION 1..11: note = FR4
WGQGTLVTVSS

SEQ ID NO: 32 - protein, 11 aa, synthetic construct
  REGION 1..11: note = FR4
WGQGTLVTVSS

SEQ ID NO: 33 - protein, 11 aa, synthetic construct
  REGION 1..11: note = FR4
SSQGTLVTVSS

SEQ ID NO: 34 - protein, 5 aa, synthetic construct
  REGION 1..5: note = CDR1
INFYG

SEQ ID NO: 35 - protein, 5 aa, synthetic construct
  REGION 1..5: note = CDR1
LNSMG

SEQ ID NO: 36 - protein, 5 aa, synthetic construct
  REGION 1..5: note = CDR1
GNVMG

SEQ ID NO: 37 - protein, 5 aa, synthetic construct
  REGION 1..5: note = CDR1
SFGMS

SEQ ID NO: 38 - protein, 16 aa, synthetic construct
  REGION 1..16: note = CDR2
HISIGDQTDYADSAKG

SEQ ID NO: 39 - protein, 16 aa, synthetic construct
  REGION 1..16: note = CDR2
IITSDGDTNYADSVKG

SEQ ID NO: 40 - protein, 16 aa, synthetic construct
  REGION 1..16: note = CDR2
AIAEGGSILYRDSVKG

SEQ ID NO: 41 - protein, 17 aa, synthetic construct
  REGION 1..17: note = CDR2
SISGSGSDTLYADSVKG

SEQ ID NO: 42 - protein, 9 aa, synthetic construct
  REGION 1..9: note = CDR3
LSRIWPYDY

SEQ ID NO: 43 - protein, 13 aa, synthetic construct
  REGION 1..13: note = CDR3
TYSTSSYSFPINT

SEQ ID NO: 44 - protein, 7 aa, synthetic construct
  REGION 1..7: note = CDR3
HPPVLPY

SEQ ID NO: 45 - protein, 6 aa, synthetic construct
  REGION 1..6: note = CDR3
GGSLSR

SEQ ID NO: 46 - protein, 30 aa, synthetic construct
  REGION 1..30: note = FR1
DVQLVESGGGVVQPGGSLRLSCVASGYVHK

SEQ ID NO: 47 - protein, 30 aa, synthetic construct
  REGION 1..30: note = FR1
EVQLVESGGGVVQPGGSLRLSCAASGGVFR

SEQ ID NO: 48 - protein, 30 aa, synthetic construct
  REGION 1..30: note = FR1
EVQLVESGGGVVQPGGSLRLSCAASGVIFS

SEQ ID NO: 49 - protein, 30 aa, synthetic construct
  REGION 1..30: note = FR1
EVQLVESGGGVVQPGGSLRLSCAASGFTFR

SEQ ID NO: 50 - protein, 14 aa, synthetic construct
  REGION 1..14: note = FR2
WYRQAPGKEREKVA

SEQ ID NO: 51 - protein, 14 aa, synthetic construct
  REGION 1..14: note = FR2
WFRQAPGKQRELVA

SEQ ID NO: 52 - protein, 15 aa, synthetic construct
  REGION 1..15: note = FR2
WYRRQAPGKEREWVA

SEQ ID NO: 53 - protein, 14 aa, synthetic construct
  REGION 1..14: note = FR2
WVRQAPGKGPEWVS

SEQ ID NO: 54 - protein, 32 aa, synthetic construct
  REGION 1..32: note = FR3
RFTISRDESKNTVYLQMNSLRPEDTAAYYCRA

SEQ ID NO: 55 - protein, 32 aa, synthetic construct
  REGION 1..32: note = FR3
RFTISRDQAKNTVYLQMNSLRPEDTALYYCQI

SEQ ID NO: 56 - protein, 32 aa, synthetic construct
  REGION 1..32: note = FR3
RFTISRDNAKNTVYLQMNSLRPEDTALYYCNS

SEQ ID NO: 57 - protein, 32 aa, synthetic construct
  REGION 1..32: note = FR3
RFTISRDNSKNTLYLQMNSLRPEDTALYYCTI

SEQ ID NO: 58 - protein, 11 aa, synthetic construct
  REGION 1..11: note = FR4
WGQGTLVTVSS

SEQ ID NO: 59 - protein, 11 aa, synthetic construct
  REGION 1..11: note = FR4
WGQGTLVTVSS

SEQ ID NO: 60 - protein, 11 aa, synthetic construct
  REGION 1..11: note = FR4
WGQGTLVTVSS

SEQ ID NO: 61 - protein, 11 aa, synthetic construct
  REGION 1..11: note = FR4
SSQGTLVTVSS

SEQ ID NO: 62 - protein, 115 aa, synthetic construct [sequence omitted]

SEQ ID NO: 63 - protein, 115 aa, synthetic construct [sequence omitted]

SEQ ID NO: 64 - protein, 116 aa, synthetic construct
  REGION 1..116: note = Alb129
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 65 - protein, 116 aa, synthetic construct
  REGION 1..116: note = Alb132
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 66 - protein, 115 aa, synthetic construct [sequence omitted]

SEQ ID NO: 67 - protein, 116 aa, synthetic construct
  REGION 1..116: note = Alb11 (S112K)-A
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 68 - protein, 115 aa, synthetic construct
  REGION 1..115: note = Alb82
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 69 - protein, 116 aa, synthetic construct
  REGION 1..116: note = Alb82-A
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 70 - protein, 117 aa, synthetic construct
  REGION 1..117: note = Alb82-AA
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 71 - protein, 118 aa, synthetic construct
  REGION 1..118: note = Alb82-AAA
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 72 - protein, 116 aa, synthetic construct
  REGION 1..116: note = Alb82-G
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 73 - protein, 117 aa, synthetic construct
  REGION 1..117: note = Alb82-GG
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 74 - protein, 118 aa, synthetic construct
  REGION 1..118: note = Alb82-GGG
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 75 - protein, 116 aa, synthetic construct
  REGION 1..116: note = Alb223
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 76 - skipped

SEQ ID NO: 77 - protein, 5 aa, synthetic construct
  REGION 1..5: note = linker
GGGGS

SEQ ID NO: 78 - protein, 7 aa, synthetic construct
  REGION 1..7: note = linker
SGGSGGS

SEQ ID NO: 79 - protein, 8 aa, synthetic construct
  REGION 1..8: note = linker
GGGGSGGS

SEQ ID NO: 80 - protein, 9 aa, synthetic construct
  REGION 1..9: note = linker
GGGGSGGGS

SEQ ID NO: 81 - protein, 10 aa, synthetic construct
  REGION 1..10: note = linker
GGGGSGGGGS

SEQ ID NO: 82 - protein, 15 aa, synthetic construct
  REGION 1..15: note = linker
GGGGSGGGGSGGGGS

SEQ ID NO: 83 - protein, 18 aa, synthetic construct
  REGION 1..18: note = linker
GGGGSGGGGSGGGGSGGS

SEQ ID NO: 84 - protein, 20 aa, synthetic construct
  REGION 1..20: note = linker
GGGGSGGGGSGGGGSGGGGS

SEQ ID NO: 85 - protein, 25 aa, synthetic construct
  REGION 1..25: note = linker
GGGGSGGGGSGGGGSGGGGSGGGGS

SEQ ID NO: 86 - protein, 30 aa, synthetic construct
  REGION 1..30: note = linker
GGGGSGGGGSGGGGSGGGGSGGGGSGGGGS

SEQ ID NO: 87 - protein, 35 aa, synthetic construct
  REGION 1..35: note = linker
GGGGSGGGGSGGGGSGGGGSGGGGSGGGGSGGGGS

SEQ ID NO: 88 - protein, 40 aa, synthetic construct
  REGION 1..40: note = linker
GGGGSGGGGSGGGGSGGGGSGGGGSGGGGSGGGGSGGGGS

SEQ ID NO: 89 - protein, 15 aa, synthetic construct
  REGION 1..15: note = linker
EPKSCDKTHTCPPCP

SEQ ID NO: 90 - protein, 24 aa, synthetic construct
  REGION 1..24: note = linker
GGGGSGGGSEPKSCDKTHTCPPCP

SEQ ID NO: 91 - protein, 12 aa, synthetic construct
  REGION 1..12: note = linker
EPKTPKPQPAAA

SEQ ID NO: 92 - protein, 62 aa, synthetic construct
  REGION 1..62: note = linker
ELKTPLGDTTHTCPRCPEPKSCDTPPPCPRCPEPKSCDTPPPCPRCPEPKSCDTPPPCPRCP

SEQ ID NO: 93 - protein, 5 aa, synthetic construct
  REGION 1..5: note = C-terminus
VTVSS

SEQ ID NO: 94 - protein, 5 aa, synthetic construct
  REGION 1..5: note = C-terminus
VKVSS

SEQ ID NO: 95 - protein, 5 aa, synthetic construct
  REGION 1..5: note = C-terminus
VQVSS

SEQ ID NO: 96 - protein, 5 aa, synthetic construct
  REGION 1..5: note = C-terminus
VTVKS

SEQ ID NO: 97 - protein, 5 aa, synthetic construct
  REGION 1..5: note = C-terminus
VTVQS

SEQ ID NO: 98 - protein, 5 aa, synthetic construct
  REGION 1..5: note = C-terminus
VKVKS

SEQ ID NO: 99 - protein, 5 aa, synthetic construct
  REGION 1..5: note = C-terminus
VKVQS

SEQ ID NO: 100 - protein, 5 aa, synthetic construct
  REGION 1..5: note = C-terminus
VQVKS

SEQ ID NO: 101 - protein, 5 aa, synthetic construct
  REGION 1..5: note = C-terminus
VQVQS

SEQ ID NO: 102 - protein, 6 aa, synthetic construct
  REGION 1..6: note = C-terminus
VTVSSA

SEQ ID NO: 103 - protein, 6 aa, synthetic construct
  REGION 1..6: note = C-terminus
VKVSSA

SEQ ID NO: 104 - protein, 6 aa, synthetic construct
  REGION 1..6: note = C-terminus
VQVSSA

SEQ ID NO: 105 - protein, 6 aa, synthetic construct
  REGION 1..6: note = C-terminus
VTVKSA

SEQ ID NO: 106 - protein, 6 aa, synthetic construct
  REGION 1..6: note = C-terminus
VTVQSA

SEQ ID NO: 107 - protein, 6 aa, synthetic construct
  REGION 1..6: note = C-terminus
VKVKSA

SEQ ID NO: 108 - protein, 6 aa, synthetic construct
  REGION 1..6: note = C-terminus
VKVQSA

SEQ ID NO: 109 - protein, 6 aa, synthetic construct
  REGION 1..6: note = C-terminus
VQVKSA

SEQ ID NO: 110 - protein, 6 aa, synthetic construct
  REGION 1..6: note = C-terminus
VQVQSA

SEQ ID NO: 111 - protein, 4 aa, synthetic construct
  REGION 1..4: note = motif
KERE

SEQ ID NO: 112 - protein, 4 aa, synthetic construct
  REGION 1..4: note = motif
KQRE

SEQ ID NO: 113 - protein, 4 aa, synthetic construct
  REGION 1..4: note = motif
GLEW

SEQ ID NO: 114 - protein, 5 aa, synthetic construct
  REGION 1..5: note = motif
KEREL

SEQ ID NO: 115 - protein, 5 aa, synthetic construct
  REGION 1..5: note = motif
KEREF

SEQ ID NO: 116 - protein, 5 aa, synthetic construct
  REGION 1..5: note = motif
KQREL

SEQ ID NO: 117 - protein, 5 aa, synthetic construct
  REGION 1..5: note = motif
KQREF

SEQ ID NO: 118 - protein, 5 aa, synthetic construct
  REGION 1..5: note = motif
KEREG

SEQ ID NO: 119 - protein, 5 aa, synthetic construct
  REGION 1..5: note = motif
KQREW

SEQ ID NO: 120 - protein, 5 aa, synthetic construct
  REGION 1..5: note = motif
KQREG

SEQ ID NO: 121 - protein, 4 aa, synthetic construct
  REGION 1..4: note = motif
TERE

SEQ ID NO: 122 - protein, 5 aa, synthetic construct
  REGION 1..5: note = motif
TEREL

SEQ ID NO: 123 - protein, 4 aa, synthetic construct
  REGION 1..4: note = motif
TQRE

SEQ ID NO: 124 - protein, 5 aa, synthetic construct
  REGION 1..5: note = motif
TQREL

SEQ ID NO: 125 - protein, 4 aa, synthetic construct
  REGION 1..4: note = motif
KECE

SEQ ID NO: 126 - protein, 5 aa, synthetic construct
  REGION 1..5: note = motif
KECEL

SEQ ID NO: 127 - protein, 5 aa, synthetic construct
  REGION 1..5: note = motif
KECER

SEQ ID NO: 128 - protein, 4 aa, synthetic construct
  REGION 1..4: note = motif
KQCE

SEQ ID NO: 129 - protein, 5 aa, synthetic construct
  REGION 1..5: note = motif
KQCEL

SEQ ID NO: 130 - protein, 4 aa, synthetic construct
  REGION 1..4: note = motif
RERE

SEQ ID NO: 131 - protein, 5 aa, synthetic construct
  REGION 1..5: note = motif
REREG

SEQ ID NO: 132 - protein, 4 aa, synthetic construct
  REGION 1..4: note = motif
RQRE

SEQ ID NO: 133 - protein, 5 aa, synthetic construct
  REGION 1..5: note = motif
RQREL

SEQ ID NO: 134 - protein, 5 aa, synthetic construct
  REGION 1..5: note = motif
RQREF

SEQ ID NO: 135 - protein, 5 aa, synthetic construct
  REGION 1..5: note = motif
RQREW

SEQ ID NO: 136 - protein, 4 aa, synthetic construct
  REGION 1..4: note = motif
QERE

SEQ ID NO: 137 - protein, 5 aa, synthetic construct
  REGION 1..5: note = motif
QEREG

SEQ ID NO: 138 - protein, 4 aa, synthetic construct
  REGION 1..4: note = motif
QQRE

SEQ ID NO: 139 - protein, 5 aa, synthetic construct
  REGION 1..5: note = motif
QQREW

SEQ ID NO: 140 - protein, 5 aa, synthetic construct
  REGION 1..5: note = motif
QQREL

SEQ ID NO: 141 - protein, 5 aa, synthetic construct
  REGION 1..5: note = motif
QQREF

SEQ ID NO: 142 - protein, 4 aa, synthetic construct
  REGION 1..4: note = motif
KGRE

SEQ ID NO: 143 - protein, 5 aa, synthetic construct
  REGION 1..5: note = motif
KGREG

SEQ ID NO: 144 - protein, 4 aa, synthetic construct
  REGION 1..4: note = motif
KDRE

SEQ ID NO: 145 - protein, 5 aa, synthetic construct
  REGION 1..5: note = motif
KDREV

SEQ ID NO: 146 - protein, 5 aa, synthetic construct
  REGION 1..5: note = motif
DECKL

SEQ ID NO: 147 - protein, 5 aa, synthetic construct
  REGION 1..5: note = motif
NVCEL

SEQ ID NO: 148 - protein, 4 aa, synthetic construct
  REGION 1..4: note = motif
GVEW

SEQ ID NO: 149 - protein, 4 aa, synthetic construct
  REGION 1..4: note = motif
EPEW

SEQ ID NO: 150 - protein, 4 aa, synthetic construct
  REGION 1..4: note = motif
GLER

SEQ ID NO: 151 - protein, 4 aa, synthetic construct
  REGION 1..4: note = motif
DQEW

SEQ ID NO: 152 - protein, 4 aa, synthetic construct
  REGION 1..4: note = motif
DLEW

SEQ ID NO: 153 - protein, 4 aa, synthetic construct
  REGION 1..4: note = motif
GIEW

SEQ ID NO: 154 - protein, 4 aa, synthetic construct
  REGION 1..4: note = motif
ELEW

SEQ ID NO: 155 - protein, 4 aa, synthetic construct
  REGION 1..4: note = motif
GPEW

SEQ ID NO: 156 - protein, 4 aa, synthetic construct
  REGION 1..4: note = motif
EWLP

SEQ ID NO: 157 - protein, 4 aa, synthetic construct
  REGION 1..4: note = motif
GPER

SEQ ID NO: 158 - protein, 4 aa, synthetic construct
  REGION 1..4: note = motif
GLER

SEQ ID NO: 159 - protein, 4 aa, synthetic construct
  REGION 1..4: note = motif
ELEW

(19) United States
(12) Patent Application Publication
(10) Pub. No.: US 2026/0285973 A1
(43) Pub. Date: Sep. 24, 2026

(54) POLYPEPTIDES COMPRISING IMMUNOGLOBULIN SINGLE VARIABLE DOMAINS TARGETING TCRaß, CD33 AND CD123

(71) Applicants: ABLYNX NV, Zwijnaarde (BE); SANOFI, Paris (FR)

(72) Inventors: Helène Bonnevaux, Neuilly-Plaisance (FR); Melissa Dullaers, Destelbergen (BE); Annelies Roobrouck, Oudenaarde (BE); Stephanie Staelens, Wevelgem (BE); Diane Van Hoorick, Laarne (BE); Judith Verhelst, Heusden (BE)

(73) Assignees: ABLYNX NV, Zwijnaarde (BE); SANOFI, Paris (FR)

(21) Appl. No.: 18/718,590

(22) PCT Filed: Dec. 16, 2022
(86) PCT No.: PCT/EP2022/086353

§ 371 Date: Jun. 11, 2024

(30) Foreign Application Priority Data
Dec. 17, 2021 (EP) 21306822.4
Apr. 7, 2022 (EP) 22305477.6

(51) Int. Cl. C07K 16/28; A61P 35/02; C07K 16/18

(52) U.S. Cl. CPC C07K 16/2803; A61P 35/02; C07K 16/18; C07K 16/2809; C07K 16/2866; C07K 2317/62; C07K 2317/92; C07K 2317/94

(57) ABSTRACT

The present technology aims at providing a novel type of drug for treating a subject suffering from acute myeloid leukemia (AML). Specifically, the present technology provides polypeptides comprising at least three immunoglobulin single variable domains (ISVDs), characterized in that at least one ISVDs binds to T cell receptor αβ (TCRαβ), at least ISVD binds to CD33, and at least one ISVD binds to CD123. The present technology also provides nucleic acids, vectors and compositions.

### RELATED APPLICATION

This application is a national stage filing under 35 U.S.C. 371 of International Patent Application Serial No. PCT/EP2022/086353, filed Dec. 16, 2022, the entire content of which is incorporated herein by reference in its entirety.

### REFERENCE TO AN ELECTRONIC SEQUENCE LISTING

The contents of the electronic sequence listing (A084870232US00-SEQ JRV.xml; Size: 198,388 bytes; and Date of Creation: Jun. 3, 2024) is herein incorporated by reference in its entirety.

### DESCRIPTION

### 1 Field of the Present Technology

The present technology relates to polypeptides targeting TCRαβ, CD33, and CD123. It also relates to nucleic acid molecules encoding these polypeptides as well as vectors comprising these nucleic acids. The present technology further relates to compositions comprising such polypeptides, nucleic acids or vectors. The present technology also relates to such compositions for use in a method of treating a subject suffering from acute myeloid leukemia (AML). Moreover, the present technology relates to methods for producing these compositions.

### 2 Technological Background

Until today the treatment of acute myeloid leukemia (AML) remains challenging. Since cytotoxic T cells appear to have highest potential for treatment of malignant diseases among all immune cells, AML treatment approaches are aiming at directing cytotoxic T cells to AML cells. The CD33 and the CD123 antigen were found to be overexpressed on blast and leukemic stem cells of most cases of AML and were thus used as suitable tumor-associated target antigens for antibody-based therapies. Mylotarg (Gemtuzumab Ozogamicin; an antiCD33-antibody drug-conjugate) was the first targeted compound registered for the treatment of AML. Recent therapies are based on dual specific antibody constructs targeting one of the tumor antigens CD33 or CD123 on the AML cells and CD3 on cytotoxic T cells. Thereby, dual-specific antibodies can anchor on CD33+ or CD123+ AML cells and at the same time can bind to CD3 on T cells. In this way, the T cells are brought into close proximity to the tumor cell. Multiple binding of the dual-specific antibodies to the tumor antigen (CD33 or CD123) on the tumor cell and simultaneous binding to the TCR-associated CD3 molecules on T cells leads to TCR clustering. This ultimately results in efficient T cell activation irrespective of TCR specificity. Cytotoxic T cell activation near AML cells may then result in tumor cell killing. Dual specific antibody constructs presently tested in clinical trials are, e.g., Flotetuzumab (MGD006; CD3/CD123 DART), AMG330 or AMG673 (both CD3/CD33 BiTEs). Effective treatment of AML is complicated given the heterogeneity of CD33 and CD123 expression that is found both within the AML patient population (inter-patient) as well as within the AML blast population of the individual patient (intra-patient). Furthermore, targeting a single tumor antigen poses the risk of losing expression of this antigen on tumor cells due to the selection pressure induced by the therapeutic intervention (Gardner et al., Blood, 127(20), 2406-2410 (2016); Blood. 2017 Jan. 5; 129(1):100-104). Thus, there is a strong need for new therapeutic approaches that overcome the limitations of the single targeting therapy and have broader patient coverage.

### 3 SUMMARY OF THE PRESENT TECHNOLOGY

The present inventors found that a polypeptide (or ISVD construct) dual targeting CD33 and CD123 on acute myeloid leukemia (AML) cells combined with targeting the T cell receptor αβ (TCR αβ) on T cells leads to efficacious killing of AML cells. The killing activity on CD33/CD123 double expressing cells was comparable to single targeting benchmarks such as CD33/CD3 AMG 330 BiTE or CD123/CD3 MGD006 DART. However, the polypeptide of the invention showed robust killing activity on CD33 and CD123 single expressing cells, whereas the single targeting benchmarks showed only activity against the cells expressing their specific target. Moreover, the polypeptide of the invention induces similar or even lower levels of inflammatory cytokines compared to benchmarks.

In some embodiments, the polypeptides of the present technology are efficiently produced (e.g. in microbial hosts) and showed low viscosity at high concentrations which is advantageous and convenient for subcutaneous administration. Furthermore, such polypeptides have limited reactivity to pre-existing antibodies in the subject to be treated (i.e. antibodies present in the subject before the first treatment with the antibody construct). In preferred embodiments such polypeptides exhibit a half-life in the subject to be treated that is long enough such that consecutive treatments can be conveniently spaced apart.

The polypeptide of the present technology comprises or consists of at least three immunoglobulin single variable domains (ISVDs), wherein at least one ISVD specifically binds to TCRαβ, at least one ISVD specifically binds to CD33 and at least one ISVD specifically bind to CD123 (an exemplary polypeptide is illustrated in FIG. 1). Preferably, the at least one ISVD binding to TCRαβ specifically binds to human TCRαβ, the at least one ISVD binding to CD33 specifically binds to human CD33 and the at least one ISVD binding to CD123 specifically binds to human CD123.

The polypeptide preferably further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units. For example, the binding unit can be an ISVD that binds to a serum protein, preferably to a human serum protein such as human serum albumin.

Also provided is a nucleic acid molecule capable of expressing the polypeptide of the present technology, a nucleic acid or vector comprising the nucleic acid, and a composition comprising the polypeptide, the nucleic acid or the vector. The composition is preferably a pharmaceutical composition.

Provided is also a host or host cell comprising the nucleic acid or vector that encodes the polypeptide according to the present technology.

Further provided is a method for producing the polypeptide according to present technology, said method at least comprising the steps of:
a. expressing, optionally in a suitable host cell or host organism or in another suitable expression system, a nucleic acid sequence encoding the polypeptide according to the present technology optionally followed by:
b. isolating and/or purifying the polypeptide according to the present technology.

Moreover, the present technology provides the polypeptide, the composition comprising the polypeptide, or the composition comprising the nucleic acid or vector comprising the nucleotide sequence that encodes the polypeptide, for use as a medicament. Preferably, the polypeptide or composition is for use in the treatment of acute myeloid leukemia (AML), wherein preferably the AML is relapsed and/or refractory AML.

In addition, provided is a method of treating AML, wherein said method comprises administering, to a subject in need thereof, a pharmaceutically active amount of the polypeptide or a composition according to the present technology. The AML is preferably relapsed and/or refractory AML. In a preferred embodiment, the method further comprises administering one or more additional therapeutic agents.

Further provided is the use of the polypeptide or composition of the present technology in the preparation of a pharmaceutical composition for treating AML, wherein AML is preferably relapsed and/or refractory AML.

In particular, the present technology provides the following embodiments:

Embodiment 1. A polypeptide, a composition comprising the polypeptide, or a composition comprising a nucleic acid comprising a nucleotide sequence that encodes the polypeptide, for use as a medicament, wherein the polypeptide comprises or consists of at least three immunoglobulin single variable domains (ISVDs), wherein each of said ISVDs comprises three complementarity determining regions (CDR1 to CDR3, respectively), optionally linked via one or more peptidic linkers; and wherein:
a) a first ISVD specifically binds to T cell receptor αβ (TCRαβ) and comprises
   i. a CDR1 which has the amino acid sequence of SEQ ID NO: 6 or has 2 or 1 amino acid difference(s) with SEQ ID NO: 6;
   ii. a CDR2 which has the amino acid sequence of SEQ ID NO: 10 or has 2 or 1 amino acid difference(s) with SEQ ID NO: 10; and
   iii. a CDR3 which has the amino acid sequence of SEQ ID NO: 14 or has 2 or 1 amino acid difference(s) with SEQ ID NO: 14;
b) a second ISVD specifically binds to CD33 and comprises
   iv. a CDR1 which has the amino acid sequence of SEQ ID NO: 7 or has 2 or 1 amino acid difference(s) with SEQ ID NO: 7;
   v. a CDR2 which has the amino acid sequence of SEQ ID NO: 11 or has 2 or 1 amino acid difference(s) with SEQ ID NO: 11; and
   vi. a CDR3 which has the amino acid sequence of SEQ ID NO: 15 or has 2 or 1 amino acid difference(s) with SEQ ID NO: 15; and
c) a third ISVD specifically binds to CD123 and comprises
   vii. a CDR1 which has the amino acid sequence of SEQ ID NO: 8 or has 2 or 1 amino acid difference(s) with SEQ ID NO: 8;
   viii. a CDR2 which has the amino acid sequence of SEQ ID NO: 12 or has 2 or 1 amino acid difference(s) with SEQ ID NO: 12; and
   ix. a CDR3 which has the amino acid sequence of SEQ ID NO: 16 or has 2 or 1 amino acid difference(s) with SEQ ID NO: 16,
wherein the ISVDs are in the order starting from the N-terminus.

Embodiment 2. The composition for use according to embodiment 1, which is a pharmaceutical composition which further comprises at least one pharmaceutically acceptable carrier, diluent or excipient and/or adjuvant, and optionally comprises one or more further pharmaceutically active polypeptides and/or compounds.

Embodiment 3. The polypeptide or composition for use according to embodiment 1 or 2, wherein:
a) said first ISVD comprises a CDR1 having the amino acid sequence of SEQ ID NO:6, a CDR2 having the amino acid sequence of SEQ ID NO: 10 and a CDR3 having the amino acid sequence of SEQ ID NO: 14;
b) said second ISVD comprises a CDR1 having the amino acid sequence of SEQ ID NO:7, a CDR2 having the amino acid sequence of SEQ ID NO: 11 and a CDR3 having the amino acid sequence of SEQ ID NO: 15; and
c) said third ISVD comprises a CDR1 having the amino acid sequence of SEQ ID NO:8, a CDR2 having the amino acid sequence of SEQ ID NO: 12 and a CDR3 having the amino acid sequence of SEQ ID NO: 16.

Embodiment 4. The polypeptide or composition for use according to any of embodiments 1 to 3, wherein:
a) the amino acid sequence of said first ISVD has a sequence identity of more than 90% with SEQ ID NO: 2;
b) the amino acid sequence of said second ISVD has a sequence identity of more than 90% with SEQ ID NO: 3; and
c) the amino acid sequence of said third ISVD has a sequence identity of more than 90% identity with SEQ ID NO: 4.

Embodiment 5. The polypeptide or composition for use according to any of embodiments 1 to 4, wherein:
a) said first ISVD has the amino acid sequence of SEQ ID NO: 2;
b) said second ISVD has the amino acid sequence of SEQ ID NO: 3; and
c) said third ISVD has the amino acid sequence of SEQ ID NO: 4.

Embodiment 6. The polypeptide or composition for use according to any of embodiments 1 to 5, wherein said polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units.

Embodiment 7. The polypeptide or composition for use according to embodiment 6, in which said one or more other groups, residues, moieties or binding units that provide the polypeptide with increased half-life is chosen from the group consisting of a polyethylene glycol molecule, serum proteins or fragments thereof, binding units that can bind to serum proteins, an Fc portion, and small proteins or peptides that can bind to serum proteins.

Embodiment 8. The polypeptide or composition for use according to any one of embodiments 6 to 7, in which said one or more other groups, residues, moieties or binding units that provide the polypeptide with increased half-life is chosen from the group consisting of binding units that can bind to serum albumin (such as human serum albumin) or a serum immunoglobulin (such as IgG).

Embodiment 9. The polypeptide or composition for use according to embodiment 8, in which said binding unit that provides the polypeptide with increased half-life is an ISVD that can bind to human serum albumin.

Embodiment 10. The polypeptide or composition for use according to embodiment 9, wherein the ISVD binding to human serum albumin comprises
i. a CDR1 which has the amino acid sequence of SEQ ID NO: 9 or has 2 or 1 amino acid difference(s) with SEQ ID NO: 9;
ii. a CDR2 which has the amino acid sequence of SEQ ID NO: 13 or has 2 or 1 amino acid difference(s) with SEQ ID NO: 13; and
iii. a CDR3 which has the amino acid sequence of SEQ ID NO: 17 or has 2 or 1 amino acid difference(s) with SEQ ID NO: 17.

Embodiment 11. The polypeptide or composition for use according to any of embodiments 9 to 10, wherein the ISVD binding to human serum albumin comprises a CDR1 having the amino acid sequence of SEQ ID NO: 9, a CDR2 having the amino acid sequence of SEQ ID NO: 13 and a CDR3 having the amino acid sequence of SEQ ID NO: 17.

Embodiment 12. The polypeptide or composition for use according to any of embodiments 9 to 11, wherein the amino acid sequence of said ISVD binding to human serum albumin has a sequence identity of more than 90% with SEQ ID NO: 5.

Embodiment 13. The polypeptide or composition for use according to any of embodiments 9 to 12, wherein said ISVD binding to human serum albumin has the amino acid sequence of SEQ ID NO: 5.

Embodiment 14. The polypeptide or composition for use according to any of embodiments 1 to 13, wherein the amino acid sequence of the polypeptide has a sequence identity of more than 90% with SEQ ID NO: 1.

Embodiment 15. The polypeptide or composition for use according to any of embodiments 1 to 14, wherein the polypeptide comprises or consists of the amino acid sequence of SEQ ID NO: 1.

Embodiment 16. The polypeptide or composition for use according to any of claims 1 to 15, for use in the treatment of AML.

Embodiment 17. The polypeptide or composition for use according to claim 16, wherein the AML is relapsed and/or refractory AML.

Embodiment 18. A polypeptide that comprises or consists of at least three immunoglobulin single variable domains (ISVDs), wherein each of said ISVDs comprises three complementarity determining regions (CDR1 to CDR3, respectively), optionally linked via one or more peptidic linkers; and wherein:
a) a first ISVD specifically binds to T cell receptor αβ (TCRαβ) and comprises
   i. a CDR1 which has the amino acid sequence of SEQ ID NO: 6 or has 2 or 1 amino acid difference(s) with SEQ ID NO: 6;
   ii. a CDR2 which has the amino acid sequence of SEQ ID NO: 10 or has 2 or 1 amino acid difference(s) with SEQ ID NO: 10; and
   iii. a CDR3 which has the amino acid sequence of SEQ ID NO: 14 or has 2 or 1 amino acid difference(s) with SEQ ID NO: 14;
b) a second ISVD specifically binds to CD33 and comprises
   iv. a CDR1 which has the amino acid sequence of SEQ ID NO: 7 or has 2 or 1 amino acid difference(s) with SEQ ID NO: 7;
   v. a CDR2 which has the amino acid sequence of SEQ ID NO: 11 or has 2 or 1 amino acid difference(s) with SEQ ID NO: 11; and
   vi. a CDR3 which has the amino acid sequence of SEQ ID NO: 15 or has 2 or 1 amino acid difference(s) with SEQ ID NO: 15; and
c) a third ISVD specifically binds to CD123 and comprises
   vii. a CDR1 which has the amino acid sequence of SEQ ID NO: 8 or has 2 or 1 amino acid difference(s) with SEQ ID NO: 8;
   viii. a CDR2 which has the amino acid sequence of SEQ ID NO: 12 or has 2 or 1 amino acid difference(s) with SEQ ID NO: 12; and
   ix. a CDR3 which has the amino acid sequence of SEQ ID NO: 16 or has 2 or 1 amino acid difference(s) with SEQ ID NO: 16,
wherein the ISVDs are in the order starting from the N-terminus.

Embodiment 19. The polypeptide according to embodiment 18, wherein:
a) said first ISVD comprises a CDR1 having the amino acid sequence of SEQ ID NO:6, a CDR2 having the amino acid sequence of SEQ ID NO: 10 and a CDR3 having the amino acid sequence of SEQ ID NO: 14;
b) said second ISVD comprises a CDR1 having the amino acid sequence of SEQ ID NO:7, a CDR2 having the amino acid sequence of SEQ ID NO: 11 and a CDR3 having the amino acid sequence of SEQ ID NO: 15; and
c) said third ISVD comprises a CDR1 having the amino acid sequence of SEQ ID NO:8, a CDR2 having the amino acid sequence of SEQ ID NO: 12 and a CDR3 having the amino acid sequence of SEQ ID NO: 16.

Embodiment 20. The polypeptide according to any of embodiments 18 or 19, wherein:
a) the amino acid sequence of said first ISVD has a sequence identity of more than 90% with SEQ ID NO: 2;
b) the amino acid sequence of said second ISVD has a sequence identity of more than 90% with SEQ ID NO: 3; and
c) the amino acid sequence of said third ISVD has a sequence identity of more than 90% identity with SEQ ID NO: 4.

Embodiment 21. The polypeptide according to any of embodiments 18 to 20, wherein:
a) said first ISVD has the amino acid sequence of SEQ ID NO: 2;
b) said second ISVD has the amino acid sequence of SEQ ID NO: 3; and
c) said third ISVD has the amino acid sequence of SEQ ID NO: 4.

Embodiment 22. The polypeptide according to any of embodiments 18 to 21, wherein said polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units.

Embodiment 23. The polypeptide according to embodiment 22, in which said one or more other groups, residues, moieties or binding units that provide the polypeptide with increased half-life is chosen from the group consisting of a polyethylene glycol molecule, serum proteins or fragments thereof, binding units that can bind to serum proteins, an Fc portion, and small proteins or peptides that can bind to serum proteins.

Embodiment 24. The polypeptide according to any one of embodiments 22 to 23, in which said one or more other groups, residues, moieties or binding units that provide the polypeptide with increased half-life is chosen from the group consisting of binding units that can bind to serum albumin (such as human serum albumin) or a serum immunoglobulin (such as IgG).

Embodiment 25. The polypeptide according to embodiment 24, in which said binding unit that provides the polypeptide with increased half-life is an ISVD that can bind to human serum albumin.

Embodiment 26. The polypeptide according to embodiment 25, wherein the ISVD binding to human serum albumin comprises
i. a CDR1 which has the amino acid sequence of SEQ ID NO: 9 or has 2 or 1 amino acid difference(s) with SEQ ID NO: 9;
ii. a CDR2 which has the amino acid sequence of SEQ ID NO: 13 or has 2 or 1 amino acid difference(s) with SEQ ID NO: 13; and
iii. a CDR3 which has the amino acid sequence of SEQ ID NO: 17 or has 2 or 1 amino acid difference(s) with SEQ ID NO: 17.

Embodiment 27. The polypeptide according to any of embodiments 25 to 26, wherein the ISVD binding to human serum albumin comprises a CDR1 having the amino acid sequence of SEQ ID NO:9, a CDR2 having the amino acid sequence of SEQ ID NO: 13 and a CDR3 having the amino acid sequence of SEQ ID NO: 17.

Embodiment 28. The polypeptide according to any of embodiments 25 to 27, wherein the amino acid sequence of said ISVD binding to human serum albumin has a sequence identity of more than 90% with SEQ ID NO: 5.

Embodiment 29. The polypeptide according to any of embodiments 25 to 28, wherein said ISVD binding to human serum albumin has the amino acid sequence of SEQ ID NO: 5.

Embodiment 30. The polypeptide according to any of embodiments 18 to 29, wherein the amino acid sequence of the polypeptide has a sequence identity of more than 90% with SEQ ID NO: 1.

Embodiment 31. The polypeptide according to any of embodiments 18 to 29, wherein the polypeptide comprises or consists of the amino acid sequence of SEQ ID NO: 1.

Embodiment 32. A nucleic acid comprising a nucleotide sequence that encodes a polypeptide according to any of embodiments 18 to 31.

Embodiment 33. A host or host cell comprising a nucleic acid according to embodiment 32.

Embodiment 34. A method for producing a polypeptide according to any of embodiments 18-31, said method at least comprising the steps of:
a) expressing, in a suitable host cell or host organism or in another suitable expression system, a nucleic acid according to embodiment 32; optionally followed by:
b) isolating and/or purifying the polypeptide according to any of embodiments 18 to 31.

Embodiment 35. A composition comprising at least one polypeptide according to any of embodiments 18 to 31, or a nucleic acid according to embodiment 32.

Embodiment 36. The composition according to embodiment 35, which is a pharmaceutical composition which further comprises at least one pharmaceutically acceptable carrier, diluent or excipient and/or adjuvant, and optionally comprises one or more further pharmaceutically active polypeptides and/or compounds.

Embodiment 37. A method of treating AML, wherein said method comprises administering, to a subject in need thereof, a pharmaceutically active amount of a polypeptide according to any of claims 18 to 31 or a composition according to any of claims 35 to 36.

Embodiment 38. The method according to claim 37, wherein the AML is relapsed and/or refractory AML.

Embodiment 39. Use of a polypeptide according to any of claims 18 to 31 or a composition according to any of claims 35 to 36, in the preparation of a pharmaceutical composition for treating AML.

Embodiment 40. Use of the polypeptide or a composition according to claim 39, wherein the AML is relapsed and/or refractory AML.

### 4 BRIEF DESCRIPTION OF THE DRAWINGS

FIG. 1: Schematic presentation of the multispecific ISVD construct according to the invention showing from the N-terminus to the C-terminus the monovalent building blocks/ISVDs TCRαβ, CD33, CD123, and Alb connected via linkers.

FIG. 2: Binding of the monovalent CD33 (left) and CD123 binding BB (right) to human (top) or cynomolgus (bottom) transfected CD33, respectively CD123 cells.

FIG. 3: Binding of A025001562 (TCR-CD33-CD123 multispecific ISVD construct, SEQ ID NO.: 1) to human CD33 and/or human CD123 expressing cells.

FIG. 4: Dose dependent inhibition of A025001562, TCR-CD33-CD123 multispecific ISVD construct, SEQ ID NO.: 1 (black squares) and reference TCR (grey dots) in the competition assays on primary T cells in the absence (dotted curves) or presence (full curves) of clinical grade HSA.

FIG. 5: Dose dependent human (top) or cynomolgus (bottom) T cell mediated killing of corresponding species CD33 (left) or CD123 (right) transfected cells, using an effector to target ratio of 15 to 1 in an impedance-based assay (xCELLigence) in the presence of 50 μM HSA.

FIG. 6: Dose dependent human (left) or cynomolgus (right) T cell mediated MOLM-13 cell killing in a flow cytometry-based assay using an effector to target ratio of 10:1. The % TO-PRO®-3 positive target cells is plotted against the concentration of the ISVD.

FIG. 7: Dose dependent human T cell mediated cell killing using an effector to target ratio of 15 to 1 in an impedance-based assay (xCELLigence). The cell index (CI) after 32-35 h of incubation is plotted against the concentration of the ISVDs.

FIG. 8: A025001562 (TCR-CD33-CD123 multispecific ISVD construct, SEQ ID NO.: 1) inhibition of Molm13-luc AML tumor growth by in vivo bioluminescence imaging.

FIG. 9: A025001562 (TCR-CD33-CD123 multispecific ISVD construct, SEQ ID NO.: 1) inhibition of Molm13-luc AML tumor growth by ex vivo bioluminescence imaging.

FIG. 10: Dose dependent human T cell mediated killing of the ISVDs according to the invention compared to CD123 and CD33 positive controls in MOLM-13 cells.

FIG. 11: Dose dependent human T cell mediated killing of the ISVDs according to the invention compared to CD123 and CD33 positive controls in KG-1a cells.

FIG. 12: Dose dependent human T cell mediated killing of the ISVDs according to the invention compared to CD123 and CD33 positive controls in U-937 cells.

FIG. 13: Dose dependent monocyte depletion of the ISVD according to the invention compared to CD123 and CD33 positive controls as well as a negative control (untargeted TCE) within human peripheral blood mononuclear cells (PBMCs).

FIG. 14: Dose dependent cytokine release of the ISVD according to the invention compared to CD123 and CD33 positive controls as well as a negative control (untargeted TCE) in human PBMCs from a healthy donor using a panel of different cytokines. A. IL-6. B. IFNγ. C. TNFα. D. IL-2.

FIG. 15: AML blast killing of the ISVD according to the invention versus CD33 and CD123 positive controls in all tested patients.

FIG. 16: Scatter plot depicting the percentage of CD33 or CD123 positive cells per AML sample.

FIG. 17: Cell viability of primary blasts from AML patients with a broad range of disease subtypes to the ISVD according to the invention compared to CD123 and CD33 positive controls as well as a negative control. A. patient number 3. B. patient number 4. C. patient number 5.

FIG. 18: Individual absolute cell counts of total CD123+ T cells (A), monocytes CD33+ cells (B), CD4+ T cells (C) and CD8+ T cells (D) as measured in peripheral blood of cynomolgus monkey treated with the ISVD according to the invention over time. Animals M1 and M2 received 0.04 μg/kg, while M3 and M4 received 0.4 μg/kg of a single 1-hour continuous intravenous infusion of a solution of the ISVD according to the invention.

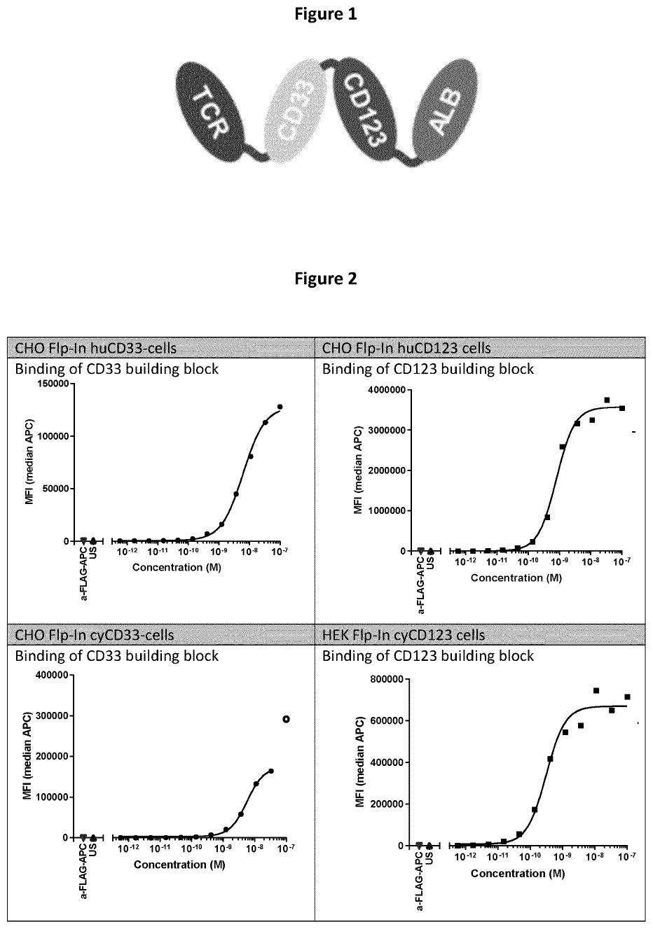

### 5 DETAILED DESCRIPTION OF THE PRESENT TECHNOLOGY

The present technology aims at providing a novel type of drug for treating acute myeloid leukemia (AML).

The present inventors found that a polypeptide (or ISVD construct) dual targeting of CD33 and CD123 on acute myeloid leukemia (AML) cells combined with targeting the T cell receptor αβ (TCR αβ) on T cells leads to efficacious killing of AML cells. The killing activity was comparable to or even higher compared to single targeting benchmarks such as CD33/CD3 AMG 330 BiTE or CD123/CD3 MGD006 DART. Due to the high heterogeneity of CD33 and CD123 expression on AML cells in intra- and inter-patient samples, the polypeptide of the present invention provides a broader patient coverage compared to the single-targeting benchmarks.

In some embodiments, the polypeptides of the present technology are efficiently produced (e.g. in microbial hosts) and showed low viscosity at high concentrations which is advantageous and convenient for subcutaneous administration. Furthermore, such polypeptides have limited reactivity to pre-existing antibodies in the subject to be treated (i.e. antibodies present in the subject before the first treatment with the antibody construct). In preferred embodiments such polypeptides exhibit a half-life in the subject to be treated that is long enough such that consecutive treatments can be conveniently spaced apart.

The polypeptide is at least bispecific, but can also be e.g., trispecific, tetraspecific or pentaspecific. Moreover, the polypeptide is at least tetravalent, but can also be e.g. pentavalent or hexavalent, etc.

The terms “bispecific”, “trispecific”, “tetraspecific”, or “pentaspecific” all fall under the term “multispecific” and refer to binding to two, three, four or five different target molecules, respectively. The terms “bivalent”, “trivalent”, “tetravalent”, “pentavalent”, or “hexavalent” all fall under the term “multivalent” and indicate the presence of two, three, four or five binding units (such as ISVDs), respectively. For example, the polypeptide may be tetraspecific-tetravalent, such as a polypeptide comprising or consisting of four ISVDs, wherein one ISVD binds to human TCRαβ, one ISVD binds to human CD33, one ISVD binds to CD123 and one ISVD binds to human serum albumin (such as ISVD construct set forth in SEQ ID NO: 1). A polypeptide may at the same time be biparatopic, for example if two ISVDs bind two different epitopes on the same target, e.g., if two ISVDs bind to TCRαβ. The term “biparatopic” refers to binding to two different parts (e.g., epitopes) of the same target molecule.

The terms “first ISVD”, “second ISVD”, “third ISVD”, etc., as used herein only indicate the relative position of the ISVDs to each other, wherein the numbering is started from the N-terminus of the polypeptide of the invention. The “first ISVD” is thus closer to the N-terminus than the “second ISVD”, whereas the “second ISVD” is closer to the N-terminus than the “third ISVD”, etc. Accordingly, the ISVD arrangement is inverse when considered from the C-terminus. Since the numbering is not absolute and only indicates the relative position of the at least three ISVDs it is not excluded that other binding units/building blocks such as additional ISVDs binding to TCRαβ, CD33, or CD123, or ISVDs binding to another target may be present in the polypeptide. For instance, as described further below (see in particular, section “(In vivo) half-life extension”), the polypeptide can further comprise another ISVD binding to human serum albumin that can be a fourth ISVD located C-terminally to the at least three ISVDs. Moreover, it does not exclude the possibility that other binding units/building blocks such as ISVDs can be placed in between. For instance, the polypeptide can further comprise another ISVD that can even be located between e.g., the “second ISVD” and “third ISVD”.

In light of the above, the invention provides a polypeptide comprising or consisting of at least three ISVDs, wherein at least one ISVD specifically binds to TCRαβ, at least one ISVD specifically binds to CD33 and at least one ISVD specifically binds to CD123.

The components, preferably ISVDs, of the polypeptide may be linked to each other by one or more suitable linkers, such as peptidic linkers.

The use of linkers to connect two or more (poly) peptides is well known in the art. Exemplary peptidic linkers are shown in Table A-5. One often used class of peptidic linker are known as the “Gly-Ser” or “GS” linkers. These are linkers that essentially consist of glycine (G) and serine(S) residues, and usually comprise one or more repeats of a peptide motif such as the GGGGS (SEQ ID NO: 77) motif (for example, having the formula (Gly-Gly-Gly-Gly-Ser), in which n may be 1, 2, 3, 4, 5, 6, 7 or more). Some often used examples of such GS linkers are 9GS linkers (GGGGSGGGS, SEQ ID NO: 80) 15GS linkers (n=3) and 35GS linkers (n=7). Reference is for example made to Chen et al., Adv. Drug Deliv. Rev. 2013 Oct. 15; 65(10): 1357-1369; and Klein et al., Protein Eng. Des. Sel. (2014) 27 (10): 325-330. In the polypeptide of the invention, the use of 9GS linkers to link the components of the polypeptide to each other is preferred.

In a preferred embodiment, the ISVD specifically binding to TCRαβ is positioned at the N-terminus of the polypeptide. The inventors surprisingly found that such a configuration can increase the production yield of the polypeptide.

Also, in a preferred embodiment, the ISVD specifically binding to CD33 is positioned C-terminally to the ISVD specifically binding to TCRαβ.

In an even more preferred embodiment, ISVD specifically binding to CD123 is positioned C-terminally to the ISVD specifically binding to CD33, which is itself C-terminally positioned to the ISVD specifically binding to TCRαβ.

Accordingly, it is preferred that the polypeptide comprises or consists of the following, in the order starting from the N-terminus of the polypeptide: a first ISVD specifically binding to TCRαβ, a second ISVD specifically binding to CD33 and a third ISVD specifically binding to CD123, and an optional binding unit providing the polypeptide with increased half-life as defined herein. The binding unit providing the polypeptide with increased half-life is preferably an ISVD.

It is even more preferred that the polypeptide comprises or consists of the following, in the order starting from the N-terminus of the polypeptide: an ISVD specifically binding to TCRαβ, a linker, an ISVD specifically binding to CD33, a linker, an ISVD specifically binding to CD123, a linker, and an ISVD binding to human serum albumin. More specifically, the polypeptide comprises or consists of the following, in the order starting from the N-terminus of the polypeptide: an ISVD specifically binding to TCRαβ, a 9GS linker, an ISVD specifically binding to CD33, a 9GS linker, an ISVD specifically binding to CD123, a 20GS linker, and an ISVD binding to human serum albumin.

Such configurations of the polypeptide can provide for increased production yield, good CMC characteristics as well as optimized functionality and stronger potency with regard to modulation of an immune response.

Preferably, the polypeptide of the present technology exhibits reduced binding by pre-existing antibodies in human serum. To this end, in one embodiment, the polypeptide comprises a valine (V) at amino acid position 11 and a leucine (L) at amino acid position 89 (according to Kabat numbering) in at least one ISVD, but preferably in each ISVD. In another embodiment, the polypeptide comprises an extension of 1 to 5 (preferably naturally occurring) amino acids, such as a single alanine (A) extension, at the C-terminus of the C-terminal ISVD. The C-terminus of an ISVD is normally VTVSS (SEQ ID NO: 93). In another embodiment the polypeptide comprises a lysine (K) or glutamine (Q) at position 110 (according to Kabat numbering) in at least one ISVD. In another embodiment, the ISVD comprises a lysine (K) or glutamine (Q) at position 112 (according to Kabat numbering) in at least one ISVD. In these embodiments, the C-terminus of the ISVD is VKVSS (SEQ ID NO: 94), VQVSS (SEQ ID NO: 95), VTVKS (SEQ ID NO: 96), VTVQS (SEQ ID NO: 97), VKVKS (SEQ ID NO: 98), VKVQS (SEQ ID NO: 99), VQVKS (SEQ ID NO: 100), or VQVQS (SEQ ID NO: 101) such that after addition of a single alanine the C-terminus of the polypeptide for example comprises the sequence VTVSSA (SEQ ID NO: 102), VKVSSA (SEQ ID NO: 103), VQVSSA (SEQ ID NO: 104), VTVKSA (SEQ ID NO: 105), VTVQSA (SEQ ID NO: 106), VKVKSA (SEQ ID NO: 107), VKVQSA (SEQ ID NO: 108), VQVKSA (SEQ ID NO: 109), or VQVQSA (SEQ ID NO: 110), preferably VTVSSA (SEQ ID NO: 102). In another embodiment, the polypeptide comprises a valine (V) at amino acid position 11 and a leucine (L) at amino acid position 89 (according to Kabat numbering) in each ISVD, optionally a lysine (K) or glutamine (Q) at position 110 (according to Kabat numbering) in at least one ISVD, and comprises an extension of 1 to 5 (preferably naturally occurring) amino acids, such as a single alanine (A) extension, at the C-terminus of the C-terminal ISVD (such that the C-terminus of the polypeptide for example comprises the sequence VTVSSA (SEQ ID NO: 102), VKVSSA (SEQ ID NO: 103) or VQVSSA (SEQ ID NO: 104), preferably VTVSSA (SEQ ID NO: 102)). See e.g. WO2012/175741 and WO2015/173325 for further information in this regard.

In a preferred embodiment, the polypeptide of the invention comprises or consists of an amino acid sequence having a sequence identity of more than 90%, such as more than 95% or more than 99%, with SEQ ID NO: 1, wherein the CDRs of the four ISVDs are as defined in items A to D (or A′ to D′ if using the Kabat definition) set forth in sections “Immunoglobulin single variable domains” and “(In vivo) half-life extension” below, respectively, wherein in particular:
the ISVD specifically binding to TCRαβ has a CDR1 having the amino acid sequence of SEQ ID NO: 6, a CDR2 having the amino acid sequence of SEQ ID NO: 10 and a CDR3 having the amino acid sequence of SEQ ID NO: 14;
the ISVD specifically binding to CD33 has a CDR1 having the amino acid sequence of SEQ ID NO: 7, a CDR2 having the amino acid sequence of SEQ ID NO: 11 and a CDR3 having the amino acid sequence of SEQ ID NO: 15;
the ISVD specifically binding to CD123 has a CDR1 having the amino acid sequence of SEQ ID NO: 8, a CDR2 having the amino acid sequence of SEQ ID NO: 12 and a CDR3 having the amino acid sequence of SEQ ID NO: 16; and
the ISVD binding to human serum albumin has a CDR1 having the amino acid sequence of SEQ ID NO: 9, a CDR2 having the amino acid sequence of SEQ ID NO: 13 and a CDR3 having the amino acid sequence of SEQ ID NO: 17,
   or alternatively if using the Kabat definition:
the ISVD specifically binding to TCRαβ has a CDR1 having the amino acid sequence of SEQ ID NO: 34, a CDR2 having the amino acid sequence of SEQ ID NO: 38 and a CDR3 having the amino acid sequence of SEQ ID NO: 42;
the ISVD specifically binding to CD33 has a CDR1 having the amino acid sequence of SEQ ID NO: 35, a CDR2 having the amino acid sequence of SEQ ID NO: 39 and a CDR3 having the amino acid sequence of SEQ ID NO: 43;
the ISVD specifically binding to CD123 has a CDR1 having the amino acid sequence of SEQ ID NO: 36, a CDR2 having the amino acid sequence of SEQ ID NO: 40 and a CDR3 having the amino acid sequence of SEQ ID NO: 44; and
the ISVD binding to human serum albumin has a CDR1 having the amino acid sequence of SEQ ID NO: 37, a CDR2 having the amino acid sequence of SEQ ID NO: 41 and a CDR3 having the amino acid sequence of SEQ ID NO: 45.

In particular, the polypeptide preferably comprises or consists of the amino acid sequence of SEQ ID NO: 1. In a most preferred embodiment, the polypeptide consists of the amino acid sequence of SEQ ID NO: 1.

The polypeptide of the invention preferably has at least half the binding affinity, more preferably at least the same binding affinity, to human TCRαβ, human CD33 and to human CD123 as compared to a polypeptide consisting of the amino acid of SEQ ID NO: 1 wherein the binding affinity is measured using the same method, such as SPR.

### 5.1 Immunoglobulin Single Variable Domains

The term “immunoglobulin single variable domain” (ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′) 2, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. Preferably, it is a V_{HH}, including a camelized V_{H} or humanized V_{HH}. Heavy chain ISVDs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) (as defined herein, and including but not limited to a NANOBODY® ISVD); other single variable domains, or any suitable fragment of any one thereof.

In particular, the immunoglobulin single variable domain may be a NANOBODY® ISVD, including a humanized V_{HH} or camelized V_{H}, or a suitable fragment thereof. [Note: NANOBODY®, NANOBODIES® and NANOCLONE® are registered trademarks of Ablynx N.V.] “V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. Nature 363:446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans (Reviews in Molecular Biotechnology 74:277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V.

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries e.g. by phage display.

The generation of immunoglobulin sequences, such as NANOBODY® ISVDs, has been described extensively in various publications, among which WO 94/04678, Hamers-Casterman et al. 1993 and Muyldermans et al. 2001 (Reviews in Molecular Biotechnology 74:277-302, 2001) can be exemplified. In these methods, camelids are immunized with the target antigen in order to induce an immune response against said target antigen. The repertoire of NANOBODY® ISVDs obtained from said immunization is further screened for ISVDs that bind the target antigen.

In these instances, the generation of antibodies requires purified antigen for immunization and/or screening. Antigens can be purified from natural sources, or in the course of recombinant production.

Immunization and/or screening for immunoglobulin sequences can be performed using peptide fragments of such antigens.

The invention may use immunoglobulin sequences of different origin, comprising mouse, rat, rabbit, donkey, human and camelid immunoglobulin sequences. The invention also includes fully human, humanized or chimeric sequences. For example, the invention comprises camelid immunoglobulin sequences and humanized camelid immunoglobulin sequences, or camelized domain antibodies, e.g. camelized dAb as described by Ward et al (see for example WO 94/04678 and Riechmann, Febs Lett., 339:285-290, 1994 and Prot. Eng., 9:531-537, 1996). Moreover, the invention also uses fused immunoglobulin sequences, e.g. forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221), and immunoglobulin sequences comprising tags or other functional moieties, e.g. toxins, labels, radiochemicals, etc., which are derivable from the immunoglobulin sequences of the present invention.

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art (e.g. WO 2008/020079).

Again, it should be noted that such humanized V_{HH}s can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein and the prior art (e.g. WO 2008/020079). Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized V_{H} is preferably a V_{H} sequence from a mammal, more preferably the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

It should be noted that one or more immunoglobulin sequences may be linked to each other and/or to other amino acid sequences (e.g. via disulphide bridges) to provide peptide constructs that may also be useful in the invention (for example Fab′ fragments, F(ab′)2 fragments, scFv constructs, “diabodies” and other multispecific constructs). Reference is for example made to the review by Holliger and Hudson, Nat Biotechnol. 2005 September; 23(9):1126-36)). Generally, when a polypeptide is intended for administration to a subject (for example for prophylactic, therapeutic and/or diagnostic purposes), it preferably comprises an immunoglobulin sequence that does not occur naturally in said subject.

A preferred structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

As further described in paragraph q) on pages 58 and 59 of WO 08/020079 (incorporated herein by reference), the amino acid residues of an immunoglobulin single variable domain can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to the AbM numbering as described in Kontermann and Dübel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods. In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence or V_{HH} sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the invention may contain one or more of hallmark residues (as defined herein), such that the ISVD sequence is a NANOBODY® immunoglobulin variable domain, including a humanized V_{HH} or camelized V_{H}. Some preferred, but non-limiting examples of (suitable combinations of) such framework sequences will become clear from the further disclosure herein.

Again, as generally described herein for the immunoglobulin sequences, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived).

However, it should be noted that the invention is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

As described above, an ISVD may be a NANOBODY® V_{HH} or a suitable fragment thereof. For a general description of NANOBODY® ISVDs, reference is made to the further description below, as well as to the prior art cited herein. In this respect, it should however be noted that this description and the prior art mainly described NANOBODY® ISVDs of the so-called “V_{H}3 class” (i.e. NANOBODY® ISVDs with a high degree of sequence homology to human germline sequences of the V_{H}3 class such as DP-47, DP-51 or DP-29). It should however be noted that the invention in its broadest sense can generally use any type of NANOBODY® ISVD, and for example also uses the NANOBODY® ISVDs belonging to the so-called “V_{H}4 class” (i.e. NANOBODY® ISVDs with a high degree of sequence homology to human germline sequences of the V_{H}4 class such as DP-78), as for example described in WO 2007/118670. Generally, NANOBODY® ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a NANOBODY® ISVD can be defined as an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a NANOBODY® ISVD can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a NANOBODY® ISVD can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table 1 below.

TABLE 1
Hallmark Residues in NANOBODY ® ISVDs

| Position | Human V_{H}3 | Hallmark Residues |
|---|---|---|
| 11 | L, V; predominantly L | L, S, V, M, W, F, T, Q, E, A, R, G, K, Y, N, P, I; |
| | | preferably L |
| 37 | V, I, F; usually V | F⁽¹⁾, Y, V, L, A, H, S, I, W, C, N, G, D, T, P, preferably |
| | | F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or |
| | | Q⁽³⁾ |
| 45⁽⁸⁾ | L | L⁽²⁾, R⁽³⁾, P, H, F, G, Q, S, E, T, Y, C, I, D, V; preferably |
| | | L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, |
| | | N, D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | p⁽⁵⁾, S, H, L, A, V, I, T, F, D, R, Y, N, Q, G, E; preferably |
| | | P |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or L⁽⁷⁾ |
| | L | |

Notes:
⁽¹⁾ In particular, but not exclusively, in combination with KERE or KQRE at positions 43-46.
⁽²⁾ Usually as GLEW at positions 44-47.
⁽³⁾ Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾ With both GLEW at positions 44-47 and KERE or KQRE at positions 43-46.
⁽⁵⁾ Often as KP or EP at positions 83-84 of naturally occurring VHH domains.
⁽⁶⁾ In particular, but not exclusively, in combination with GLEW at positions 44-47.
⁽⁷⁾ With the proviso that when positions 44-47 are GLEW, position 108 is always Q in (non-humanized) VHH sequences that also contain a W at 103.
⁽⁸⁾ The GLEW group also contains GLEW-like sequences at positions 44-47, such as for example GVEW, EPEW, GLER, DQEW, DLEW, GIEW, ELEW, GPEW, EWLP, GPER, GLER and ELEW.

The present technology inter alia uses ISVDs that can specifically bind to TCRαβ, CD33, or CD123. In the context of the present technology, “binding to” a certain target molecule has the usual meaning in the art as understood in the context of antibodies and their respective antigens.

The polypeptide of the present technology may comprise one or more ISVDs specifically binding to TCRαβ, one or more ISVDs specifically binding to CD33 and one or more ISVDs specifically binding to CD123.

The ISVDs used in the present technology form part of a polypeptide of the present technology, which comprises or consists of at least three ISVDs, such that the polypeptide can specifically bind to TCRαβ, CD33, and CD123. Thereby, the polypeptide can anchor on CD33+CD123+ leukemia stem cells (LSCs) and acute myeloid leukemia (AML) blasts and at the same time can bind to TCRαβ on cytotoxic T cells. In this way the polypeptide brings the T cells into close proximity to the LSCs and AML blasts. Multiple binding of the polypeptides to tumor antigens (CD33 and CD123) on the tumor cell and simultaneous binding to TCR molecules on a single T cell leads to TCR clustering and ultimately to T cell activation. T cell activation in proximity to the LSCs and AML blasts may lead to efficient tumor cell killing and consequently to therapeutic efficacy in AML patients.

Accordingly, the target molecules of the at least three ISVDs as used in the polypeptide of the present technology are TCRαβ, CD33, and CD123. Examples are mammalian CD33, CD123 and TCRαβ. While human TCRαβ (Uniprot accession), human CD33 (Uniprot accession) and human CD123 (Uniprot accession) are preferred, the versions from other species are also amenable to the present technology, for example TCRαβ, CD33, and CD123 from mice, rats, rabbits, cats, dogs, goats, sheep, horses, pigs, non-human primates, such as cynomolgus monkeys (also referred to herein as “cyno”), or camelids, such as llama or alpaca.

Specific examples of ISVDs specifically binding to TCRαβ, CD123 and CD123 that can be used in the present technology are as described in the following items A to C:

A. An ISVD that specifically binds to human TCRαβ and comprises
i. a CDR1 which has the amino acid sequence of SEQ ID NO: 6 or has 2 or 1 amino acid difference with SEQ ID NO: 6;
ii. a CDR2 which has the amino acid sequence SEQ ID NO: 10 or has 2 or 1 amino acid difference with SEQ ID NO: 10; and
iii. a CDR3 which has the amino acid sequence of SEQ ID NO: 14 or has 2 or 1 amino acid difference with SEQ ID NO: 14,
preferably a CDR1 having the amino acid sequence of SEQ ID NO: 6, a CDR2 having the amino acid sequence of SEQ ID NO: 10 and a CDR3 having the amino acid sequence of SEQ ID NO: 14.

Preferred examples of such an ISVD that specifically binds to human TCRαβ have one or more, and preferably all, framework regions as indicated for the TCRαβ-V_{HH} in Table A-2 (in addition to the CDRs as defined in the preceding item A), and most preferred is an ISVD having the full amino acid sequence of TCRαβ-V_{HH} (SEQ ID NO: 2, see Table A-1 and A-2). Also, in a preferred embodiment, the amino acid sequence of the ISVD specifically binding to human TCRαβ may have a sequence identity of more than 90%, such as more than 95% or more than 99%, with SEQ ID NO: 2, wherein optionally the CDRs are as defined in the preceding item A. In particular, the ISVD specifically binding to TCRαβ preferably has the amino acid sequence of SEQ ID NO: 2.

When such an ISVD specifically binding to TCRαβ has 2 or 1 amino acid difference in at least one CDR relative to a corresponding reference CDR sequence (item A above), the ISVD preferably has at least half the binding affinity, more preferably at least the same binding affinity, to human TCRαβ as TCRαβ-V_{HH} (SEQ ID NO: 2), wherein the binding affinity is measured using the same method, such as SPR.

B. An ISVD that specifically binds to human CD33 and comprises
i. a CDR1 which has the amino acid sequence SEQ ID NO: 7 or has 2 or 1 amino acid difference with SEQ ID NO: 7;
ii. a CDR2 which has the amino acid sequence SEQ ID NO: 11 or has 2 or 1 amino acid difference with SEQ ID NO: 11; and
iii. a CDR3 which has the amino acid sequence SEQ ID NO: 15 or has 2 or 1 amino acid difference with SEQ ID NO: 15,
preferably a CDR1 having the amino acid sequence of SEQ ID NO: 7, a CDR2 having the amino acid sequence of SEQ ID NO: 11 and a CDR3 having the amino acid sequence of SEQ ID NO: 15.

Preferred examples of such an ISVD that specifically binds to human CD33 have one or more, and preferably all, framework regions as indicated for CD33-V_{HH} in Table A-2 (in addition to the CDRs as defined in the preceding item B), and most preferred is an ISVD having the full amino acid sequence of CD33-V_{HH} (SEQ ID NO: 3, see Table A-1 and A-2).

Also, in a preferred embodiment, the amino acid sequence of an ISVD specifically binding to human CD33 may have a sequence identity of more than 90%, such as more than 95% or more than 99%, with SEQ ID NO: 3, wherein optionally the CDRs are as defined in the preceding item B. In particular, the ISVD binding to CD33 preferably has the amino acid sequence of SEQ ID NO: 3.

When such an ISVD specifically binding to CD33 has 2 or 1 amino acid difference in at least one CDR relative to a corresponding reference CDR sequence (item B above), the ISVD preferably has at least half the binding affinity, more preferably at least the same binding affinity, to human CD33 as construct CD33-V_{HH} (SEQ ID NO: 3), wherein the binding affinity is measured using the same method, such as SPR.

C. An ISVD that specifically binds to human CD123 and comprises
i. a CDR1 which has the amino acid sequence of SEQ ID NO: 8 or has 2 or 1 amino acid difference with SEQ ID NO: 8;
ii. a CDR2 which has the amino acid sequence of SEQ ID NO: 12 or has 2 or 1 amino acid difference with SEQ ID NO: 12; and
iii. a CDR3 which has the amino acid sequence of SEQ ID NO: 16 or has 2 or 1 amino acid difference with SEQ ID NO: 16,
preferably a CDR1 having the amino acid sequence of SEQ ID NO: 8, a CDR2 having the amino acid sequence of SEQ ID NO: 12 and a CDR3 having the amino acid sequence of SEQ ID NO: 16.

Preferred examples of such an ISVD that specifically binds to human CD123 have one or more, and preferably all, framework regions as indicated for CD123-V_{HH} in Table A-2 (in addition to the CDRs as defined in the preceding item C), and most preferred is an ISVD having the full amino acid sequence of CD123-V_{HH} (SEQ ID NO: 4, see Table A-1 and A-2).

Also, in a preferred embodiment, the amino acid sequence of an ISVD specifically binding to human CD123 may have a sequence identity of more than 90%, such as more than 95% or more than 99%, with SEQ ID NO: 4, wherein optionally the CDRs are as defined in the preceding item C. In particular, the ISVD binding to CD123 preferably has the amino acid sequence of SEQ ID NO: 4.

When such an ISVD specifically binding to CD123 has 2 or 1 amino acid difference in at least one CDR relative to a corresponding reference CDR sequence (item C above), the ISVD preferably has at least half the binding affinity, more preferably at least the same binding affinity, to human CD123 as CD123-V_{HH} (SEQ ID NO: 4), wherein the binding affinity is measured using the same method, such as SPR.

Preferably, each of the ISVDs as defined under items A to C above is comprised in the polypeptide of the invention. Such a polypeptide of the invention comprising each of the ISVDs as defined under items A to C above preferably has at least half the binding affinity, more preferably at least the same binding affinity, to human TCRαβ, to human CD33 and to human CD123 as a polypeptide consisting of the amino acid of SEQ ID NO: 1, wherein the binding affinity is measured using the same method, such as SPR.

The SEQ ID NOs referred to in the above items A to C are based on the CDR definition according to the AbM definition (see Table A-2). It is noted that the SEQ ID NOs defining the same CDRs according to the Kabat definition (see Table A-2.1) can likewise be used in the above items A to C.

Accordingly, the specific ISVDs specifically binding to TCRαβ, CD33 or CD123 that can be used in the invention as described above using the AbM definition can be also described using the Kabat definition as set forth in items A′ to C′ below:

A′. An ISVD that specifically binds to human TCRαβ and comprises
i. a CDR1 which has the amino acid sequence of SEQ ID NO: 34 or has 2 or 1 amino acid difference with SEQ ID NO: 34;
ii. a CDR2 which has the amino acid sequence SEQ ID NO: 38 or has 2 or 1 amino acid difference with SEQ ID NO: 38; and
iii. a CDR3 which has the amino acid sequence of SEQ ID NO: 42 or has 2 or 1 amino acid difference with SEQ ID NO: 42,
preferably a CDR1 having the amino acid sequence of SEQ ID NO: 34, a CDR2 having the amino acid sequence of SEQ ID NO: 38 and a CDR3 having the amino acid sequence of SEQ ID NO: 42.

Preferred examples of such an ISVD that specifically binds to human TCRαβ have one or more, and preferably all, framework regions as indicated for TCRαβ-V_{HH} in Table A-2.1 (in addition to the CDRs as defined in the preceding item A′), and most preferred is an ISVD having the full amino acid sequence of TCRαβ-V_{HH} (SEQ ID NO: 2, see Table A-1 and A-2.1).

B′. An ISVD that specifically binds to human CD33 and comprises
i. a CDR1 which has the amino acid sequence SEQ ID NO: 35 or has 2 or 1 amino acid difference with SEQ ID NO: 35;
ii. a CDR2 which has the amino acid sequence SEQ ID NO: 39 or has 2 or 1 amino acid difference with SEQ ID NO: 39; and
iii. a CDR3 which has the amino acid sequence SEQ ID NO: 43 or has 2 or 1 amino acid difference with SEQ ID NO: 43,
preferably a CDR1 having the amino acid sequence of SEQ ID NO: 35, a CDR2 having the amino acid sequence of SEQ ID NO: 39 and a CDR3 having the amino acid sequence of SEQ ID NO: 43.

Preferred examples of such an ISVD that specifically binds to human CD33 have one or more, and preferably all, framework regions as indicated for CD33-V_{HH} in Table A-2.1 (in addition to the CDRs as defined in the preceding item B′), and most preferred is an ISVD having the full amino acid sequence of CD33-V_{HH} (SEQ ID NO: 3, see Table A-1 and A-2.1).

C′. An ISVD that specifically binds to human CD123 and comprises
i. a CDR1 which has the amino acid sequence of SEQ ID NO: 36 or has 2 or 1 amino acid difference with SEQ ID NO: 36;
ii. a CDR2 which has the amino acid sequence of SEQ ID NO: 40 or has 2 or 1 amino acid difference with SEQ ID NO: 40; and
iii. a CDR3 which has the amino acid sequence of SEQ ID NO: 44 or has 2 or 1 amino acid difference with SEQ ID NO: 44,
preferably a CDR1 having the amino acid sequence of SEQ ID NO: 36, a CDR2 having the amino acid sequence of SEQ ID NO: 40 and a CDR3 having the amino acid sequence of SEQ ID NO: 44.

Preferred examples of such an ISVD that specifically binds to human CD123 have one or more, and preferably all, framework regions as indicated for CD123-V_{HH} in Table A-2.1 (in addition to the CDRs as defined in the preceding item C′), and most preferred is an ISVD having the full amino acid sequence of CD123-V_{HH} (SEQ ID NO: 4, see Table A-1 and A-2.1).

The percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (i.e. at a single position).

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

An “amino acid difference” as used herein refers to a deletion, insertion or substitution of a single amino acid residue vis-à-vis a reference sequence, and preferably is a substitution.

Amino acid substitutions are preferably conservative substitutions. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

### 5.2 Specificity

The terms “specificity”, “binding specifically” or “specific binding” refer to the number of different target molecules, such as antigens, from the same organism to which a particular binding unit, such as an ISVD, can bind with sufficiently high affinity (see below). “Specificity”, “binding specifically” or “specific binding” are used interchangeably herein with “selectivity”, “binding selectively” or “selective binding”. Binding units, such as ISVDs, preferably specifically bind to their designated targets.

The specificity/selectivity of a binding unit can be determined based on affinity. The affinity denotes the strength or stability of a molecular interaction. The affinity is commonly given as by the KD, or dissociation constant, comprising units of mol/liter (or M). The affinity can also be expressed as an association constant, KA, which equals 1/KD and has units of (mol/liter)⁻¹ (or M⁻¹).

The affinity is a measure for the binding strength between a moiety and a binding site on the target molecule: the lesser the value of the KD, the stronger the binding strength between a target molecule and a targeting moiety.

Typically, binding units used in the present technology (such as ISVDs) will bind to their targets with a dissociation constant (KD) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (KA) of 10⁵ to 10¹² liter/moles or more, and preferably 10⁷ to 10¹² liter/moles or more and more preferably 10⁸ to 10¹² liter/moles).

Any KD value greater than 10⁻⁴ mol/liter (or any KA value lower than 104 liters/mol) is generally considered to indicate non-specific binding.

The KD for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 pM) or less.

Accordingly, specific/selective binding may mean that—using the same measurement method, e.g. SPR—a binding unit (or polypeptide comprising the same) binds to TCRαβ, CD33 and/or CD123 with a KD value of 10⁻⁵ to 10⁻¹² moles/liter or less and binds to related targets with a KD value greater than 10⁻⁴ moles/liter.

Thus, the polypeptide of the present technology preferably has at least half the binding affinity, more preferably at least the same binding affinity, to human TCRαβ, to human CD33 and to human CD123 as compared to a polypeptide consisting of the amino acid of SEQ ID NO: 1, wherein the binding affinity is measured using the same method, such as SPR.

Specific binding to a certain target from a certain species does not exclude that the binding unit can also specifically bind to the analogous target from a different species. For example, specific binding to human TCRαβ does not exclude that the binding unit (or a polypeptide comprising the same) can also specifically bind to TCRαβ from cynomolgus monkeys. Likewise, for example, specific binding to human CD33 or CD123 does not exclude that the binding unit (or a polypeptide comprising the same) can also specifically bind to CD33 or CD123 from cynomolgus monkeys (“cyno”).

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.

The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁻⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (KA), by means of the relationship [KD=1/KA].

The affinity of a molecular interaction between two molecules can be measured via different techniques known per se, such as the well-known surface plasmon resonance (SPR) biosensor technique (see for example Ober et al. 2001, Intern. Immunology 13: 1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a GE Healthcare company, Uppsala, Sweden and Piscataway, NJ). For further descriptions, see Jonsson et al. (1993, Ann. Biol. Clin. 51: 19-26), Jonsson et al. (1991 Biotechniques 11: 620-627), Johnsson et al. (1995, J. Mol. Recognit. 8: 125-131), and Johnson et al. (1991, Anal. Biochem. 198: 268-277).

Another well-known biosensor technique to determine affinities of biomolecular interactions is bio-layer interferometry (BLI) (see for example Abdiche et al. 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al. 2004, Anal. Biochem., 328: 35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

The GYROLAB® immunoassay system provides a platform for automated bioanalysis and rapid sample turnaround (Fraley et al. 2013, Bioanalysis 5: 1765-74).

### 5.3 (In Vivo) Half-Life Extension

The polypeptide may further comprise one or more other groups, residues, moieties or binding units, optionally linked via one or more peptidic linkers, in which said one or more other groups, residues, moieties or binding units provide the polypeptide with increased (in vivo) half-life, compared to the corresponding polypeptide without said one or more other groups, residues, moieties or binding units. In vivo half-life extension means, for example, that the polypeptide has an increased half-life in a mammal, such as a human subject, after administration. Half-life can be expressed for example as t½beta.

The type of groups, residues, moieties or binding units is not generally restricted and may for example be chosen from the group consisting of a polyethylene glycol molecule, serum proteins or fragments thereof, binding units that can bind to serum proteins, an Fc portion, and small proteins or peptides that can bind to serum proteins.

More specifically, said one or more other groups, residues, moieties or binding units that provide the polypeptide with increased half-life can be chosen from the group consisting of binding units that can bind to serum albumin, such as human serum albumin, or a serum immunoglobulin, such as IgG, and preferably is a binding unit that can bind to human serum albumin. The binding unit is preferably an ISVD.

For example, WO 04/041865 describes NANOBODY® ISVDs binding to serum albumin (and in particular against human serum albumin) that can be linked to other proteins (such as one or more other NANOBODY® ISVDs binding to a desired target) in order to increase the half-life of said protein.

The international application WO 06/122787 describes a number of NANOBODY® ISVDs against (human) serum albumin. These NANOBODY® ISVDs include the NANOBODY® ISVD called Alb-1 (SEQ ID NO: 52 in WO 06/122787) and humanized variants thereof, such as Alb-8 (SEQ ID NO: 62 in WO 06/122787). Again, these can be used to extend the half-life of therapeutic proteins and polypeptide and other therapeutic entities or moieties.

Moreover, WO2012/175400 describes a further improved version of Alb-1, called Alb-23.

In a preferred embodiment, the polypeptide comprises a serum albumin binding moiety selected from Alb-1, Alb-3, Alb-4, Alb-5, Alb-6, Alb-7, Alb-8, Alb-9, Alb-10 and Alb-23, preferably Alb-8 or Alb-23 or its variants, as shown on pages 7-9 of WO2012/175400 and the albumin binders described in WO2012/175741, WO2015/173325, WO2017/080850, WO2017/085172, WO2018/104444, WO2018/134235, WO2018/134234. Some preferred serum albumin binders are also shown in Table A-4. A particularly preferred further component of the polypeptide of the present technology is as described in item C:

C. An ISVD that binds to human serum albumin and comprises
i. a CDR1 comprising the amino acid sequence of SEQ ID NO: 9 or has 2 or 1 amino acid difference(s) with SEQ ID NO: 9;
ii. a CDR2 comprising the amino acid sequence of SEQ ID NO: 13 or has 2 or 1 amino acid difference(s) with SEQ ID NO: 13; and
iii. a CDR3 comprising the amino acid sequence of SEQ ID NO: 17 or has 2 or 1 amino acid difference(s) with SEQ ID NO: 17;
preferably a CDR1 comprising the amino acid sequence of SEQ ID NO: 9, a CDR2 comprising the amino acid sequence of SEQ ID NO: 13 and a CDR3 comprising the amino acid sequence of SEQ ID NO: 17.

Preferred examples of such an ISVD that binds to human serum albumin have one or more (and preferably all) framework regions as indicated for construct ALB23002 in Table A-2 (in addition to the CDRs as defined in the preceding item C), and most preferred is an ISVD comprising the full amino acid sequence of construct ALB23002 (SEQ ID NO: 5, see Table A-1 and A-2).

Item C can be also described using the Kabat definition as:

C′. An ISVD that binds to human serum albumin and comprises
i. a CDR1 comprising the amino acid sequence of SEQ ID NO: 37 or has 2 or 1 amino acid difference with SEQ ID NO: 37;
ii. a CDR2 comprising the amino acid sequence of SEQ ID NO: 41 or has 2 or 1 amino acid difference with SEQ ID NO: 41; and
iii. a CDR3 comprising the amino acid sequence of SEQ ID NO: 45 or has 2 or 1 amino acid difference with SEQ ID NO: 45;
preferably a CDR1 comprising the amino acid sequence of SEQ ID NO: 37, a CDR2 comprising the amino acid sequence of SEQ ID NO: 41 and a CDR3 comprising the amino acid sequence of SEQ ID NO: 45.

Preferred examples of such an ISVD that binds to human serum albumin have one or more, and preferably all, framework regions as indicated for construct ALB23002 in Table A-2.1 (in addition to the CDRs as defined in the preceding item C′), and most preferred is an ISVD comprising the full amino acid sequence of construct ALB23002 (SEQ ID NO: 5, see Table A-1 and A-2.1).

Also in a preferred embodiment, the amino acid sequence of an ISVD binding to human serum albumin may have a sequence identity of more than 90%, such as more than 95% or more than 99%, with SEQ ID NO: 5, wherein optionally the CDRs are as defined in the preceding item C. In particular, the ISVD binding to human serum albumin preferably comprises the amino acid sequence of SEQ ID NO: 5.

When such an ISVD binding to human serum albumin has 2 or 1 amino acid difference in at least one CDR relative to a corresponding reference CDR sequence (item C above), the ISVD has at least half the binding affinity, preferably at least the same binding affinity to human serum albumin as construct ALB23002 set forth in SEQ ID NO: 5, wherein the binding affinity is measured using the same method, such as SPR.

In a preferred embodiment, when such an ISVD binding to human serum albumin has a C-terminal position it exhibits a C-terminal alanine (A) or glycine (G) extension and is preferably selected from SEQ ID NOs: 64, 65, 67, 69, 70, 71, 72, 73, 74, and 75 (see table A-4 below). If the ISVD binding to human serum albumin comprises another position than the C-terminal position (i.e. is not the C-terminal ISVD of the polypeptide of the present technology) and is selected from SEQ ID NOs: 5, 62, 63, 66, and 68 (see table A-4 below).

### 5.4 Nucleic Acid Molecules

Also provided is a nucleic acid molecule encoding the polypeptide of the present technology. A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g. for expression and/or production of a polypeptide. Suitable hosts or host cells for production purposes will be clear to the skilled person, and may for example be any suitable fungal, prokaryotic or eukaryotic cell or cell line or any suitable fungal, prokaryotic or eukaryotic organism. A host or host cell comprising a nucleic acid encoding the polypeptide of the present technology is also encompassed by the present technology.

A nucleic acid may be for example DNA, RNA, or a hybrid thereof, and may also comprise (e.g. chemically) modified nucleotides, like PNA. It can be single- or double-stranded, and is preferably in the form of double-stranded DNA. For example, the nucleotide sequences of the present technology may be genomic DNA, cDNA.

The nucleic acids of the present technology can be prepared or obtained in a manner known per se, and/or can be isolated from a suitable natural source. Nucleotide sequences encoding naturally occurring (poly) peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### 5.5 Vectors

Also provided is a vector comprising the nucleic acid molecule encoding the polypeptide of the present technology. A vector as used herein is a vehicle suitable for carrying genetic material into a cell. A vector includes naked nucleic acids, such as plasmids or mRNAs, or nucleic acids embedded into a bigger structure, such as liposomes or viral vectors.

Vectors generally comprise at least one nucleic acid that is optionally linked to one or more regulatory elements, such as for example one or more suitable promoter(s), enhancer(s), terminator(s), etc.). The vector preferably is an expression vector, i.e. a vector suitable for expressing an encoded polypeptide or construct under suitable conditions, e.g. when the vector is introduced into a (e.g. human) cell. For DNA-based vectors, this usually includes the presence of elements for transcription (e.g. a promoter and a polyA signal) and translation (e.g. Kozak sequence).

Preferably, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Preferably, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g. a coding sequence—to which it is operably linked.

### 5.6 Compositions

The present technology also provides a composition comprising at least one polypeptide of the present technology, at least one nucleic acid molecule encoding a polypeptide of the present technology or at least one vector comprising such a nucleic acid molecule. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

### 5.7 Host Organisms

The present technology also pertains to host cells or host organisms comprising the polypeptide of the present technology, the nucleic acid encoding the polypeptide of the present technology, and/or the vector comprising the nucleic acid molecule encoding the polypeptide of the present technology.

Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

### 5.8 Methods and Uses of the Polypeptide

The present technology also provides a method for producing the polypeptide of the present technology. The method may comprise transforming/transfecting a host cell or host organism with a nucleic acid encoding the polypeptide, expressing the polypeptide in the host, optionally followed by one or more isolation and/or purification steps. Specifically, the method may comprise:
a) expressing, in a suitable host cell or host organism or in another suitable expression system, a nucleic acid sequence encoding the polypeptide; optionally followed by:
b) isolating and/or purifying the polypeptide.

Suitable host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

The polypeptide of the present technology, a nucleic acid molecule or vector as described, or a composition comprising the polypeptide of the present technology, nucleic acid molecule or vector—preferably the polypeptide or a composition comprising the same—are useful as a medicament.

Accordingly, the present technology provides the polypeptide of the present technology, a nucleic acid molecule or vector as described, or a composition comprising the polypeptide of the present technology, nucleic acid molecule or vector for use as a medicament.

Also provided is the polypeptide of the present technology, a nucleic acid molecule or vector as described, or a composition comprising the polypeptide of the present technology, nucleic acid molecule or vector for use in the (prophylactic or therapeutic) treatment of acute myeloid leukemia (AML), preferably relapsed and/or refractory AML.

Further provided is a (prophylactic and/or therapeutic) method of treating AML, wherein said method comprises administering, to a subject in need thereof, a pharmaceutically active amount of the polypeptide of the present technology, a nucleic acid molecule or vector as described, or a composition comprising the polypeptide of the present technology, nucleic acid molecule or vector.

Further provided is the use of the polypeptide of the present technology, a nucleic acid molecule or vector as described, or a composition comprising the polypeptide of the present technology, nucleic acid molecule or vector in the preparation of a pharmaceutical composition, preferably for treating AML.

The AML may be relapsed and/or refractory AML.

A “subject” as referred to in the context of the present technology can be any animal, preferably a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca).

In the context of prophylactic and/or therapeutic purposes, the subject can be any animal, and more specifically any mammal, but preferably is a human subject.

Substances (including polypeptides, nucleic acid molecules and vectors) or compositions may be administered to a subject by any suitable route of administration, for example by enteral (such as oral or rectal) or parenteral (such as epicutaneous, sublingual, buccal, nasal, intra-articular, intradermal, intramuscular, intraperitoneal, intravenous, subcutaneous, transdermal, or transmucosal) administration. Parenteral administration, such as intramuscular, subcutaneous or intradermal, administration is preferred. Most preferred is subcutaneous administration.

An effective amount of a polypeptide, a nucleic acid molecule or vector as described, or a composition comprising the polypeptide, nucleic acid molecule or vector can be administered to a subject in order to provide the intended treatment results.

One or more doses can be administered. If more than one dose is administered, the doses can be administered in suitable intervals in order to maximize the effect of the polypeptide, composition, nucleic acid molecule or vector.

TABLE A-1
Amino acid sequences of the different monovalent V_{HH} building blocks identified
within the tetravalent polypeptide A025001562 (TCR-CD33-CD123 multispecific ISVD
construct) (“ID” refers to the SEQ ID NO as used herein)

| Name | ID | Amino acid sequence |
|---|---|---|
| TCR-V_{HH} | 2 | DVQLVESGGGVVQPGGSLRLSCVASGYVHKINFYGWYRQAPGKERE |
| (building block 1) | | KVAHISIGDQTDYADSAKGRFTISRDESKNTVYLQMNSLRPEDTAAYY |
| | | CRALSRIWPYDYWGQGTLVTVSS |
| CD33-V_{HH} | 3 | EVQLVESGGGVVQPGGSLRLSCAASGGVFRLNSMGWFRQAPGKQR |
| (building block 2) | | ELVAIITSDGDTNYADSVKGRFTISRDQAKNTVYLQMNSLRPEDTALYY |
| | | CQITYSTSSYSFPINTWGQGTLVTVSS |
| CD123-V_{HH} | 4 | EVQLVESGGGVVQPGGSLRLSCAASGVIFSGNVMGWYRRQAPGKER |
| (building block 3) | | EWVAAIAEGGSILYRDSVKGRFTISRDNAKNTVYLQMNSLRPEDTALY |
| | | YCNSHPPVLPYWGQGTLVTVSS |
| ALB23002 | 5 | EVQLVESGGGVVQPGGSLRLSCAASGFTFRSFGMSWVRQAPGKGPE |
| (building block 4) | | WVSSISGSGSDTLYADSVKGRFTISRDNSKNTLYLQMNSLRPEDTALYY |
| | | CTIGGSLSRSSQGTLVTVSS |

TABLE A-2
Sequences for CDRs according to AbM numbering and frameworks (“ID” refers to the given SEQ ID NO)

| ID | V_{HH} | ID | FR1 | ID | CDR1 | ID | FR2 |
|---|---|---|---|---|---|---|---|
| 2 | TCR | 18 | DVQLVESGGGV | 6 | GYVHKIN | 22 | WYRQAPGK |
| | | | VQPGGSLRLSC | | FYG | | EREKVA |
| | | | VAS | | | | |
| 3 | CD33 | 19 | EVQLVESGGGV | 7 | GGVFRLN | 23 | WFRQAPGK |
| | | | VQPGGSLRLSC | | SMG | | QRELVA |
| | | | AAS | | | | |
| 4 | CD123 | 20 | EVQLVESGGGV | 8 | GVIFSGN | 24 | WYRRQAPG |
| | | | VQPGGSLRLSC | | VMG | | KEREWVA |
| | | | AAS | | | | |
| 5 | ALB3002 | 21 | EVQLVESGGGV | 9 | GFTFRSF | 25 | WVRQAPGK |
| | | | VQPGGSLRLSC | | GMS | | GPEWVS |
| | | | AAS | | | | |
| ID | CDR2 | ID | FR3 | ID | CDR3 | ID | FR4 |
| 10 | HISIGD | 26 | YADSAKGRFTISRDESKNTV | 14 | LSRIWPYDY | 30 | WGQGTL |
| | QTD | | YLQMNSLRPEDTAAYYCRA | | | | VTVSS |
| 11 | IITSDGD | 27 | YADSVKGRFTISRDQAKNTV | 15 | TYSTSSYSFPIN | 31 | WGQGTL |
| | TN | | YLQMNSLRPEDTALYYCQI | | T | | VTVSS |
| 12 | AIAEGG | 28 | YRDSVKGRFTISRDNAKNTV | 16 | HPPVLPY | 32 | WGQGTL |
| | SIL | | YLQMNSLRPEDTALYYCNS | | | | VTVSS |
| 13 | SISGSGS | 29 | YADSVKGRFTISRDNSKNTL | 17 | GGSLSR | 33 | SSQGTLV |
| | | | YLQMNSLRPEDTALYYCTI | | | | TVSS |

TABLE A-2.1
Sequences for CDRs according to Kabat numbering and frameworks (“ID” refers to the given SEQ ID NO)

| ID | V_{HH} | ID | FR1 | ID | CDR1 | ID | FR2 |
|---|---|---|---|---|---|---|---|
| 2 | TCR | 46 | DVQLVESGGGV | 34 | INFYG | 50 | WYRQAPGK |
| | | | VQPGGSLRLSC | | | | EREKVA |
| | | | VASGYVHK | | | | |
| 3 | CD33 | 47 | EVQLVESGGGV | 35 | LNSMG | 51 | WFRQAPGK |
| | | | VQPGGSLRLSC | | | | QRELVA |
| | | | AASGGVFR | | | | |
| 4 | CD123 | 48 | EVQLVESGGGV | 36 | GNVMG | 52 | WYRRQAPG |
| | | | VQPGGSLRLSC | | | | KEREWVA |
| | | | AASGVIFS | | | | |
| 5 | ALB3002 | 49 | EVQLVESGGGV | 37 | SFGMS | 53 | WVRQAPGK |
| | | | VQPGGSLRLSC | | | | GPEWVS |
| | | | AASGFTFR | | | | |
| ID | CDR2 | ID | FR3 | ID | CDR3 | ID | FR4 |
| 38 | HISIGDQ | 54 | RFTISRDESKNTVYLQMN | 42 | LSRIWPYDY | 58 | WGQGTL |
| | TDYADS | | SLRPEDTAAYYCRA | | | | VTVSS |
| | AKG | | | | | | |
| 39 | IITSDGD | 55 | RFTISRDQAKNTVYLQM | 43 | TYSTSSYSFPIN | 59 | WGQGTL |
| | TNYADS | | NSLRPEDTALYYCQI | | T | | VTVSS |
| | VKG | | | | | | |
| 40 | AIAEGGS | 56 | RFTISRDNAKNTVYLQM | 44 | HPPVLPY | 60 | WGQGTL |
| | ILYRDSV | | NSLRPEDTALYYCNS | | | | VTVSS |
| | KG | | | | | | |
| 41 | SISGSGS | 57 | RFTISRDNSKNTLYLQMN | 45 | GGSLSR | 61 | SSQGTLV |
| | DTLYADS | | SLRPEDTALYYCTI | | | | TVSS |
| | VKG | | | | | | |

TABLE A-3
Amino acid sequences of selected multivalent polypeptide (“ID” refers to the given SEQ ID NO)

| Name | ID | Amino acid sequence |
|---|---|---|
| A025001562 | 1 | DVQLVESGGGVVQPGGSLRLSCVASGYVHKINFYGWYRQAPGKEREKVAHISIGDQTDYADSAKGRFTISRDESKN |
| | | TVYLQMNSLRPEDTAAYYCRALSRIWPYDYWGQGTLVTVSSGGGGSGGGSEVQLVESGGGVVQPGGSLRLSCAASG |
| | | GVFRLNSMGWFRQAPGKQRELVAIITSDGDTNYADSVKGRFTISRDQAKNTVYLQMNSLRPEDTALYYCQITYSTS |
| | | SYSFPINTWGQGTLVTVSSGGGGSGGGSEVQLVESGGGVVQPGGSLRLSCAASGVIFSGNVMGWYRRQAPGKEREW |
| | | VAAIAEGGSILYRDSVKGRFTISRDNAKNTVYLQMNSLRPEDTALYYCNSHPPVLPYWGQGTLVTVSSGGGGSGGG |
| | | GSGGGGSGGGGSEVQLVESGGGVVQPGGSLRLSCAASGFTFRSFGMSWVRQAPGKGPEWVSSISGSGSDTLYADSV |
| | | KGRFTISRDNSKNTLYLQMNSLRPEDTALYYCTIGGSLSRSSQGTLVTVSSA |

TABLE A-4
Serum albumin binding ISVD sequences (“ID” refers to the SEQ ID NO as used
herein)

| Name | ID | Amino acid sequence |
|---|---|---|
| Alb8 | 62 | EVQLVESGGGLVQPGNSLRLSCAASGFTFSSFGMSWVRQAPGKGLEWV |
| | | SSISGSGSDTLYADSVKGRFTISRDNAKTTLYLQMNSLRPEDTAVYYCTIG |
| | | GSLSRSSQGTLVTVSS |
| Alb23 | 63 | EVQLLESGGGLVQPGGSLRLSCAASGFTFRSFGMSWVRQAPGKGPEWV |
| | | SSISGSGSDTLYADSVKGRFTISRDNSKNTLYLQMNSLRPEDTAVYYCTIG |
| | | GSLSRSSQGTLVTVSS |
| Alb129 | 64 | EVQLVESGGGVVQPGNSLRLSCAASGFTFSSFGMSWVRQAPGKGLEW |
| | | VSSISGSGSDTLYADSVKGRFTISRDNAKTTLYLQMNSLRPEDTATYYCTIG |
| | | GSLSRSSQGTLVTVSSA |
| Alb132 | 65 | EVQLVESGGGVVQPGGSLRLSCAASGFTFRSFGMSWVRQAPGKGPEW |
| | | VSSISGSGSDTLYADSVKGRFTISRDNSKNTLYLQMNSLRPEDTATYYCTIG |
| | | GSLSRSSQGTLVTVSSA |
| Alb11 | 66 | EVQLVESGGGLVQPGNSLRLSCAASGFTFSSFGMSWVRQAPGKGLEWV |
| | | SSISGSGSDTLYADSVKGRFTISRDNAKTTLYLQMNSLRPEDTAVYYCTIG |
| | | GSLSRSSQGTLVTVSS |
| Alb11 | 67 | EVQLVESGGGLVQPGNSLRLSCAASGFTFSSFGMSWVRQAPGKGLEWV |
| (S112K)-A | | SSISGSGSDTLYADSVKGRFTISRDNAKTTLYLQMNSLRPEDTAVYYCTIG |
| | | GSLSRSSQGTLVKVSSA |
| Alb82 | 68 | EVQLVESGGGVVQPGNSLRLSCAASGFTFSSFGMSWVRQAPGKGLEW |
| | | VSSISGSGSDTLYADSVKGRFTISRDNAKTTLYLQMNSLRPEDTALYYCTIG |
| | | GSLSRSSQGTLVTVSS |
| Alb82-A | 69 | EVQLVESGGGVVQPGNSLRLSCAASGFTFSSFGMSWVRQAPGKGLEW |
| | | VSSISGSGSDTLYADSVKGRFTISRDNAKTTLYLQMNSLRPEDTALYYCTIG |
| | | GSLSRSSQGTLVTVSSA |
| Alb82-AA | 70 | EVQLVESGGGVVQPGNSLRLSCAASGFTFSSFGMSWVRQAPGKGLEW |
| | | VSSISGSGSDTLYADSVKGRFTISRDNAKTTLYLQMNSLRPEDTALYYCTIG |
| | | GSLSRSSQGTLVTVSSAA |
| Alb82-AAA | 71 | EVQLVESGGGVVQPGNSLRLSCAASGFTFSSFGMSWVRQAPGKGLEW |
| | | VSSISGSGSDTLYADSVKGRFTISRDNAKTTLYLQMNSLRPEDTALYYCTIG |
| | | GSLSRSSQGTLVTVSSAAA |
| Alb82-G | 72 | EVQLVESGGGVVQPGNSLRLSCAASGFTFSSFGMSWVRQAPGKGLEW |
| | | VSSISGSGSDTLYADSVKGRFTISRDNAKTTLYLQMNSLRPEDTALYYCTIG |
| | | GSLSRSSQGTLVTVSSG |
| Alb82-GG | 73 | EVQLVESGGGVVQPGNSLRLSCAASGFTFSSFGMSWVRQAPGKGLEW |
| | | VSSISGSGSDTLYADSVKGRFTISRDNAKTTLYLQMNSLRPEDTALYYCTIG |
| | | GSLSRSSQGTLVTVSSGG |
| Alb82- | 74 | EVQLVESGGGVVQPGNSLRLSCAASGFTFSSFGMSWVRQAPGKGLEW |
| GGG | | VSSISGSGSDTLYADSVKGRFTISRDNAKTTLYLQMNSLRPEDTALYYCTIG |
| | | GSLSRSSQGTLVTVSSGGG |
| Alb23002 | 5 | EVQLVESGGGVVQPGGSLRLSCAASGFTFRSFGMSWVRQAPGKGPEW |
| | | VSSISGSGSDTLYADSVKGRFTISRDNSKNTLYLQMNSLRPEDTALYYCTIG |
| | | GSLSRSSQGTLVTVSS |
| Alb223 | 75 | EVQLVESGGGVVQPGGSLRLSCAASGFTFRSFGMSWVRQAPGKGPEW |
| | | VSSISGSGSDTLYADSVKGRFTISRDNSKNTLYLQMNSLRPEDTALYYCTIG |
| | | GSLSRSSQGTLVTVSSA |

TABLE A-5
Linker sequences (“ID” refers to the SEQ ID NO as used herein)

| Name | ID | Amino acid sequence |
|---|---|---|
| 3A linker | 76 | AAA |
| 5GS linker | 77 | GGGGS |
| 7GS linker | 78 | SGGSGGS |
| 8GS linker | 79 | GGGGSGGS |
| 9GS linker | 80 | GGGGSGGGS |
| 10GS linker | 81 | GGGGSGGGGS |
| 15GS linker | 82 | GGGGSGGGGSGGGGS |
| 18GS linker | 83 | GGGGSGGGGSGGGGSGGS |
| 20GS linker | 84 | GGGGSGGGGSGGGGSGGGGS |
| 25GS linker | 85 | GGGGSGGGGSGGGGSGGGGSGGGGS |
| 30GS linker | 86 | GGGGSGGGGSGGGGSGGGGSGGGGSGGGGS |
| 35GS linker | 87 | GGGGSGGGGSGGGGSGGGGSGGGGSGGGGSGGGGS |
| 40GS linker | 88 | GGGGSGGGGSGGGGSGGGGSGGGGSGGGGSGGGGSGGGGS |
| G1 hinge | 89 | EPKSCDKTHTCPPCP |
| 9GS-G1 hinge | 90 | GGGGSGGGSEPKSCDKTHTCPPCP |
| Llama upper long | 91 | EPKTPKPQPAAA |
| hinge region | | |
| G3 hinge | 92 | ELKTPLGDTTHTCPRCPEPKSCDTPPPCPRCPEPKSCDTPPPCPRC |
| | | PEPKSCDTPPPCPRCP |

### 6 EXAMPLES

### 6.1 Example 1: Multispecific ISVD Construct Generation

Multi-specific NANOBODY® ISVD proteins were expressed in P. pastoris. The yeast expression vectors contain the AOX1 promoter and terminator, a resistance gene for Zeocin and the coding information for the Saccharomyces cerevisiae a-mating factor signal peptide. The NANOBODY® ISVD monovalent building blocks (BBs) were combined with GS linkers and cloned in the expression vector via Golden Gate cloning (Engler C, Marillonnet S. Golden Gate cloning. Methods Mol Biol. 2014; 1116:119-31). The expression vectors contain two BpiI restriction sites for the cloning the PCR-amplified monovalent NANOBODY® ISVD building blocks together with the GS linkers included in one or multiple vectors. All these elements are flanked by BpiI sites. The use of unique nucleotide overhangs for each position of the cloning cassette allows seamless ligation in a pre-defined order. After Sanger sequence confirmation, plasmid DNA derived from E. coli TOP10 was linearized and transformed by electroporation into in-house prepared hypercompetent P. pastoris, strain NRRL Y-11430 (ATCC 76273).

E. coli TG1 cells (Lucigen, Cat. No. 60502), containing the NANOBODY® ISVD protein expression vector, were grown for 2 hours at 37° C. followed by 29 hours at 30° C. (250 rpm) in a baffled shaker flask containing “5052” auto-induction medium. Cells were pelleted by centrifugation (20 minutes, 4500 rpm, 4° C.), the supernatant was discarded, and pellets were frozen over night at −20° C. The frozen cell pellets were then dissolved in DPBS at 1/12.5^{th} of the original culture volume and incubated at 4° C. for 1 hour while gently rotating, to disrupt the outer membrane of the cells. The cells were pelleted again (20 minutes, 8500 rpm, 4° C.) and the supernatant, containing the NANOBODY® ISVD proteins, was collected and filtered to immediately proceed with purification. FLAG3His₆-tagged NANOBODY® ISVD proteins were purified by immobilized metal affinity chromatography (IMAC) on or NiIDA/NTA (Genscript) resins with Imidazole (for the former) or acidic elution (for the latter) followed by a desalting step (PD columns with Sephadex G25 resin, GE Healthcare) and if necessary, preparative size exclusion chromatography (SEC) (Superdex 75 column, GE Healthcare) in D-PBS. To this end, robotic stations or ÄKTA purification systems were used.

Tagless NANOBODY® ISVD proteins or constructs containing the ALB building block were purified on Amsphere A3 (JSR) or MabCaptureA (Poros) resins followed by a desalting step (PD columns with Sephadex G25 resin, GE Healthcare) and if necessary, preparative SEC (Superdex 75 column, GE Healthcare) in D-PBS. N-octyl-β-d-glucopyranoside (OGP; Alpha Aesar, Cat. No. J67390) treatment was implemented during purification/gel filtration chromatography whenever low LPS levels were required. Concentration was determined via OD280/OD340 measurement. Quality control was performed by SDS-PAGE and mass spectrometry.

### 6.2 Example 2: Multispecific ISVD Construct Binding Affinity to TCRαβ, CD33, CD123 and Serum Albumin

The TCRαβ-CD33-CD123 multispecific ISVD construct as set forth in SEQ ID NO: 1 was generated.

### 6.2.1 Affinity Determination Towards Human and Cynomolgus CD33 and CD123 Proteins

The affinities, expressed as the association rate constant (kₐ), the dissociation rate constant (k_{d}) and the equilibrium dissociation constant (K_{D}), of the TCRαβ-CD33-CD123 multispecific ISVD construct (SEQ ID NO: 1) towards recombinant huCD33L-Fc, cyCD33L-Fc (via capturing setup) or huCD123 (via capturing setup) proteins were measured at 37° C. by means of a surface plasmon resonance (SPR)-based assay on a ProteOn XPR36 instrument (BioRad Laboratories, Inc.).

### Setup for Measuring Binding Affinity Towards CD33

Anti-huIgG (Fc) was immobilized on a GLH (long matrix, high capacity) sensor chip via amine coupling, using EDC (1-Ethyl-3-(3-dimethylaminopropyl) carbodiimide) and NHS (N-hydroxysuccinimide esters) chemistry. Next, huCD33L-Fc and cyCD33L-Fc were captured (association: 120 s, 25 μL/min). Purified TCRαβ-CD33-CD123 multispecific ISVD construct as set forth in SEQ ID NO: 1 was injected at different concentrations (0.4 to 625 nM) for 120 s and dissociation was followed for 900 s.

### Setup for Measuring Binding Affinity Towards CD123

Anti-huIgG (Fc) was immobilized on a GLH (long matrix, high capacity) sensor chip via amine coupling, using EDC (1-Ethyl-3-(3-dimethylaminopropyl) carbodiimide) and NHS (N-hydroxysuccinimide esters) chemistry. Next, huCD123-Fc and cyCD123-Fc were captured (association: 120 s, 25 μL/min). Purified TCRαβ-CD33-CD123 multispecific ISVD construct as set forth in SEQ ID NO: 1 was injected at different concentrations (1.2 to 300 nM) for 120 s and dissociation was followed for 900 s, at 45 μl/min.

Data was double referenced by subtracting a reference analyte lane and a blank buffer injection. Affinity constants (kₐ, k_{d}, K_{D}) were calculated applying the Langmuir 1:1 interaction model using ProteOn Manager 3.1.0 (Version 3.1.0.6) software.

The results of the affinity measurement of TCRαβ-CD33-CD123 multispecific ISVD construct as set forth in SEQ ID NO: 1 to human and cynomolgus CD33 and human and cynomolgus CD123 are summarized in Table 2 and Table 3 below.

TABLE 2
SPR based affinity determination of TCRαβ-CD33-CD123 multispecific
ISVD construct for human and cynomolgus CD33 protein

| | Human CD33L-Fc | | | Cynomolgus CD33L-Fc | | |
|---|---|---|---|---|---|---|
| ISVD | kₐ (1/Ms) | k_{d} (1/s) | K_{D} (M) | kₐ (1/Ms) | kd (1/s) | K_{D} (M) |
| TCRαβ- | 3.5E+05 | 2.7E−03 | 7.6E−09 | 2.8E+05 | 1.9E−03 | 6.9E−09 |
| CD33- | | | | | | |
| CD123 | | | | | | |
| (SEQ ID | | | | | | |
| NO: 1) | | | | | | |

Reactivity of TCRαβ-CD33-CD123 multispecific ISVD construct towards cynomolgus CD33 (K_{D}=6.9 nM) was comparable to human CD33 (K_{D}=7.6 nM).

TABLE 3
SPR based kinetic determination of TCRαβ-CD33-CD123 multispecific
ISVD construct for human and cynomolgus CD123 protein

| | Human CD123-Fc | | | Cynomolgus CD123-Fc | | |
|---|---|---|---|---|---|---|
| ISVD | kₐ (1/Ms) | k_{d} (1/s) | K_{D} (M) | kₐ (1/Ms) | kd (1/s) | K_{D} (M) |
| TCRαβ- | 1.3E+06 | 7.4E−04 | 5.9E−10 | 1.2E+06 | 2.4E−03 | 2.0E−09 |
| CD33- | | | | | | |
| CD123 | | | | | | |
| (SEQ ID | | | | | | |
| NO: 1) | | | | | | |

Reactivity of TCRαβ-CD33-CD123 multispecific ISVD construct towards cynomolgus CD123 (K_{D}=2 nM) was 3.3-fold lower than to the human CD123 (K_{D}=0.59 nM).

The results (Table 2 and Table 3) demonstrate that the multispecific ISVD construct binds human/cynomolgus CD33 and human/cynomolgus CD123 with high affinity.

### 6.2.2 Affinity Determination Towards Human and Cynomolgus TCRαβ Protein

The affinities, expressed as the association rate constant (kₐ), the dissociation rate constant (k_{d}) and the equilibrium dissociation constant (K_{D}), of the TCRαβ-CD33-CD123 multispecific ISVD construct (SEQ ID NO: 1) towards recombinant huTCR(2XN9)-zipper, cyTCR(AEA41865)-zipper (via coated setup) were evaluated at 37° C. by means of an SPR based assay on a ProteOn XPR36 instrument (BioRad Laboratories, Inc.).

### Setup for Measuring Binding Affinity Towards TCRαβ

huTCR(2XN9)-zipper, cyTCR(AEA41865)-zipper proteins were coated a GLC (short matrix, normal capacity) sensor chip. Purified TCRαβ-CD33-CD123 multispecific ISVD construct as set forth in SEQ ID NO: 1 was injected at different concentrations (0.4 to 625 nM) for 120 s and dissociation was followed for 900 s.

Data was double referenced by subtracting a reference analyte lane and a blank buffer injection. Affinity constants (kₐ, k_{d}, K_{D}) were calculated applying the Langmuir 1:1 interaction model using ProteOn Manager 3.1.0 (Version 3.1.0.6) software.

The results of the affinity measurement of TCRαβ-CD33-CD123 multispecific ISVD construct as set forth in SEQ ID NO: 1 to human and cynomolgus TCRαβ are summarized in Table 4 below. As a reference an ISVD consisting only of the TCRαβ building block (SEQ ID NO: 2) linked to the ALB23002 (SEQ ID NO: 5) was taken along.

TABLE 4
SPR based kinetic determination of TCRαβ-CD33-CD123 multispecific ISVD
construct and reference TCR-ISVD construct for human and cynomolgus TCRαβ

| | Human TCRαβ-zipper | | | | Cynomolgus TCRαβ-zipper | | | |
|---|---|---|---|---|---|---|---|---|
| ISVD | n | kₐ (1/Ms) | k_{d} (1/s) | K_{D} (M) | n | kₐ (1/Ms) | k_{d} (1/s) | K_{D} (M) |
| TCRαβ- | 1 | 8.0E+04 | 1.7E−03 | 2.1E−08 | 1 | 4.9E+05 | 3.3E−03 | 6.7E−09 |
| CD33-CD123 | | | | | | | | |
| (SEQ ID NO: 1) | | | | | | | | |
| TCR | Experiment1 | 1.4E+05 | 1.7E−03 | 1.2E−08 | Experiment1 | 4.2E+05 | 4.4E−03 | 1.1E−08 |
| (reference) | Experiment2 | 2.6E+05 | 1.8E−03 | 7.0E−09 | Experiment2 | 8.2E+05 | 3.5E−03 | 4.3E−09 |

Reactivity of TCRαβ-CD33-CD123 multispecific ISVD construct towards cyno TCRαβ (K_{D}=6.7 nM) was 3-fold higher than to the human TCRαβ (K_{D}=21 nM.)

The results (Table 4) demonstrate that the multispecific ISVD construct binds human/cynomolgus TCRαβ with high affinity.

### 6.2.3 Affinity Determination Towards Human, Mouse and Cynomolgus Serum Albumin

Binding affinity, expressed as the association rate constant (kₐ), the dissociation rate constant (k_{d}) and the equilibrium dissociation constant (K_{D}), of the TCRαβ-CD33-CD123 multispecific ISVD construct (SEQ ID NO: 1) towards recombinant to human, cynomolgus and mouse serum albumin via the C-terminal ALB23002 half-life extension (via coated setup) were evaluated at 37° C. by means of an SPR based assay on a ProteOn XPR36 instrument (BioRad Laboratories, Inc.).

### Setup for Measuring Binding Affinity Serum Albumin

Human, cynomolgus and mouse serum albumin were immobilized on a ProteOn GLC sensor chip using amine coupling using amine coupling, using EDC and NHS chemistry (running buffer used: HBS-EP+, pH 7.4). The albumins were immobilized at two concentrations of 2.5 μg/mL (HSA & MSA) and 5 μg/mL (CSA) in pH4.5 Acetate buffer, giving rise to immobilization levels of up to 220 RUs for CSA, 150 RUs for MSA and 110 RUs for HSA. Purified multivalent V_{HH}s were injected for 2 minutes (flow rate 45 μL/min) at different concentrations (between 4.3 nM and 416 nM) and dissociation was followed for 900s. Regeneration between cycles consisted of an injection of 10 mM glycine-HCL, pH1.5 for 47 s at 100 μL/min.

Data was double referenced by subtracting a reference ligand lane and a buffer injection. Processed curves were evaluated via fitting with the Langmuir 1:1 interaction model using ProteOn Manager 3.1.0 (Version 3.1.0.6) software model and affinity constants (kₐ, k_{d}, K_{D}) were calculated.

The results of the affinity measurement of TCRαβ-CD33-CD123 multispecific ISVD construct as set forth in SEQ ID NO: 1 to human, mouse and cynomolgus serum albumin are summarized in Table 5 below.

TABLE 5
SPR based affinity determination of TCRαβ-CD33-CD123 multispecific ISVD construct for serum albumin

| | MSA | | | CSA | | | HSA | | |
|---|---|---|---|---|---|---|---|---|---|
| ISVD | kₐ (1/Ms) | k_{d} (1/s) | K_{D} (M) | kₐ (1/Ms) | k_{d} (1/s) | K_{D} (M) | kₐ (1/Ms) | k_{d} (1/s) | K_{D} (M) |

| | | | | | | | |
|---|---|---|---|---|---|---|---|
| TCRαβ- | KD < 5E−07 | 2.5E+05 | 2.0E−03 | 7.9E−09 | 2.2E+05 | 2.1E−03 | 9.7E−09 |
| CD33-CD123 | | | | | | | |
| (SEQ ID NO: 1) | | | | | | | |

Cross-reactivity towards CSA was confirmed. In addition, although no kinetic parameters were reported, affinity for MSA was good enough to obtain half-life extension and adopt the half-life of the serum albumin.

The results (Table 5) demonstrate that the multispecific ISVD construct binds human/mouse/cynomolgus TCRαβ with high affinity.

### 6.3 Example 3: Multispecific ISVD Construct Binding Affinity to Membrane Bound CD33 and/or CD123

### Setup for Measuring Binding Affinity to CD33/CD123 on Target Cell Lines

The binding affinity of TCRαβ-CD33-CD123 multispecific ISVD construct towards target cell lines expressing CD33 and/or CD123 was evaluated using flow cytometry. The target cell lines expressing CD123 are described in detail in WO2018/091606A1.

Transfected CD33 cells were generated as follows. Stable CHO Flp-In (Invitrogen, R758-07) cell lines with recombinant overexpression of CD33, were generated using the Flp-In™ site-directed recombination technology (Flp-In™ System For Generating Stable Mammalian Expression Cell Lines by Flp Recombinase-Mediated Integration (Invitrogen, K601001, K601002)). Hereby, DNA integration occurs at a specific genomic location at an FRT (Flp Recombination Target) site by the Flp recombinase (pOG44) derived from Saccharomyces cerevisiae. The Flp-In™ host cell line and expression plasmid (pcDNA5) both contain this FRT site, thereby allowing a single homologous DNA recombination. The sequence for human CD33 was derived from NCBI RefSeq NP_001763, the sequence of cynomolgus CD123 was derived from NCBI genbank no. XP_005590138.

In brief, cells were harvested and transferred to a V-bottom 96-well plate (5×10⁴ cells per well) and incubated with a serial dilution of TCRαβ-CD123-CD33 multispecific ISVD construct for 30 min at 4° C. in FACS buffer (D-PBS (Gibco, 14190) with 10% FBS (Sigma, F7524) and 0.05% sodium azide (Acros organics, 19038)) in the presence of 30 μM clinical grade HSA (CSL Behring, 2160-679) in a final volume of 100 μL. Next, cells were washed three times with FACS buffer and incubated with 1 μg/mL mouse monoclonal ANTI-FLAG® M2 antibody (Sigma-Aldrich, F1804) for 30 min at 4° C. for detection of FLAG3His₆-tagged CD123-CD33-TCR multispecific ISVD construct or with 3 μg/mL mAb anti-V_{HH} antibody (ABH0077) (APS+In House, A-0006-00_ABH0077_SF_AB1891) for detection of the NANOBODY® ISVD with an ALB BB. Next, cells were washed 3 times with FACS buffer and incubated for 30 min at 4° C. with 5 μg/mL Allophycocyanin (APC) AffiniPure Goat Anti-Mouse IgG (subclasses 1+2a+2b+3), Fcγ Fragment Specific (Jackson Immunoresearch, 115-136-071), in a final volume of 100 μL. Subsequently, cells were resuspended in 50 μL cold FACS buffer supplemented with 1 μg/ml the Propidium iodide (PI, Sigma P4170) to distinguish live from dead cells. After staining, cells were acquired using the MACSQuant X flow cytometer (Miltenyi Biotec) and analyzed using FlowLogic (Miltenyi Biotec). First a P1 population which represented more than 80% of the total events was selected based on FSC-SSC distribution, to distinguish cells from debris. From this population (P1) the PI positive (dead) cells were excluded and the median APC fluorescence intensity of the PI negative cells was evaluated.

### Setup for Measuring Binding Affinity to Primary T Cells

The binding affinity of TCRαβ-CD33-CD123 multispecific ISVD construct towards primary T cells was evaluated using flow cytometry in a competition setup using the FLAG3His₆ tagged monovalent TCR ISVD as ligand.

In brief, human or cynomolgus primary T cells were thawed and transferred to a V-bottom 96-well plate (7.5×10⁴ cells per well in 100 μL) and incubated with a serial dilution of TCRαβ-CD33-CD123 multispecific ISVD construct and a fixed concentration of ligand for 30 min at 4° C. in FACS buffer (D-PBS (Gibco, 14190) with 10% FBS (Sigma, F7524) and 0.05% sodium azide (Acros organics, 19038)) in the presence of 30 μM clinical grade HSA CSL Behring, 2160-679) in a final volume of 100 μL. The concentration of ligand used in the assay was below its binding EC₅₀. After an incubation period of 90 min at 4° C., the level of ligand binding was determined via flow cytometry. Thereto, cells were washed 3 times and incubated with 1 μg/ml mouse monoclonal ANTI-FLAG® M2 antibody (Sigma-Aldrich, F1804) for 30 min at 4° C., washed again, and incubated for 30 min at 4° C. with 5 μg/ml Allophycocyanin (APC) AffiniPure Goat Anti-Mouse IgG (subclasses 1+2a+2b+3), Fcγ fragment specific (Jackson Immunoresearch, 115-136-071), in a final volume of 100 μL. Subsequently, cells were resuspended in FACS buffer supplemented with 1 μg/ml Propidium Iodide (PI, Sigma, P4170) to distinguish live from dead cells. After staining, cells were acquired using the MACSQuant X flow cytometer (Miltenyi Biotec) and analyzed using FlowLogic (Miltenyi Biotec). First a P1 population which represented more than 80% of the total events was selected based on FSC-SSC distribution, to distinguish cells from debris. From this population (P1) the PI positive (dead) cells were excluded and the median APC fluorescence intensity of the PI negative cells was evaluated.

The human-cynomolgus cross-reactivity was evaluated by testing the binding of the monovalent CD33 building block (SEQ ID NO: 3) and the monovalent CD123 building block (SEQ ID NO: 4) to human or cynomolgus CD33 transfected cell lines or to human or cynomolgus CD123 transfected cell lines using flow cytometry as described above. The results are graphically represented in FIG. 2. The EC₅₀ values of the different experiments are summarized in Table 6 (CD33 transfected target cells) and Table 7 (CD123 transfected target cells).

TABLE 6
EC₅₀ values for binding of the CD33 building block (SEQ
ID NO: 3) to cells expressing huCD33 or cyCD33.

| | | EC50 | 95% LCI | 95% UCI |
|---|---|---|---|---|
| Sample ID | Cell line | (M) | (M) | (M) |
| CD33 binding ISVD | CHO cy CD33 | 5.7E−09 | 3.2E−09 | 5.1E−08 |
| CD33 binding ISVD | CHO cy CD33 | 1.2E−08 | 8.2E−09 | 2.2E−08 |
| CD33 binding ISVD | CHO hu CD33 | 6.2E−09 | 4.6E−09 | 8.5E−09 |
| CD33 binding ISVD | CHO hu CD33 | 5.5E−09 | 4.1E−09 | 7.7E−09 |
| CD33 binding ISVD | CHO hu CD33 | 7.7E−09 | 6.8E−09 | 8.8E−09 |

TABLE 7
EC₅₀ values for binding of the CD123 building block (SEQ
ID NO: 4) to cells expressing huCD123 or cyCD123.

| | | EC50 | 95% LCI | 95% UCI |
|---|---|---|---|---|
| Sample ID | Cell line | (M) | (M) | (M) |
| CD123 binding ISVD | CHO human CD123 | 3.1E−10 | 2.4E−10 | 4.1E−10 |
| CD123 binding ISVD | CHO human CD123 | 2.0E−10 | 2.9E−11 | undetermined |
| CD123 binding ISVD | HEK293 cyno CD123 | 8.0E−10 | 7.2E−10 | 9.1E−10 |

Binding of the monovalent CD33 and CD123 building blocks to human and cyno membrane target was confirmed. The difference in EC₅₀ between human and cynomolgus monkey was less than 2-fold decrease for binding to CD33 and 3-fold decrease for binding to CD123 cells (Table 7).

In conclusion, in addition to the binding affinity towards recombinant human and cyno CD33 and CD123 protein dose-dependent binding of TCRαβ-CD33-CD123 multispecific ISVD construct to human and cyno cell-expressed CD33 and CD123 was also confirmed (n=1).

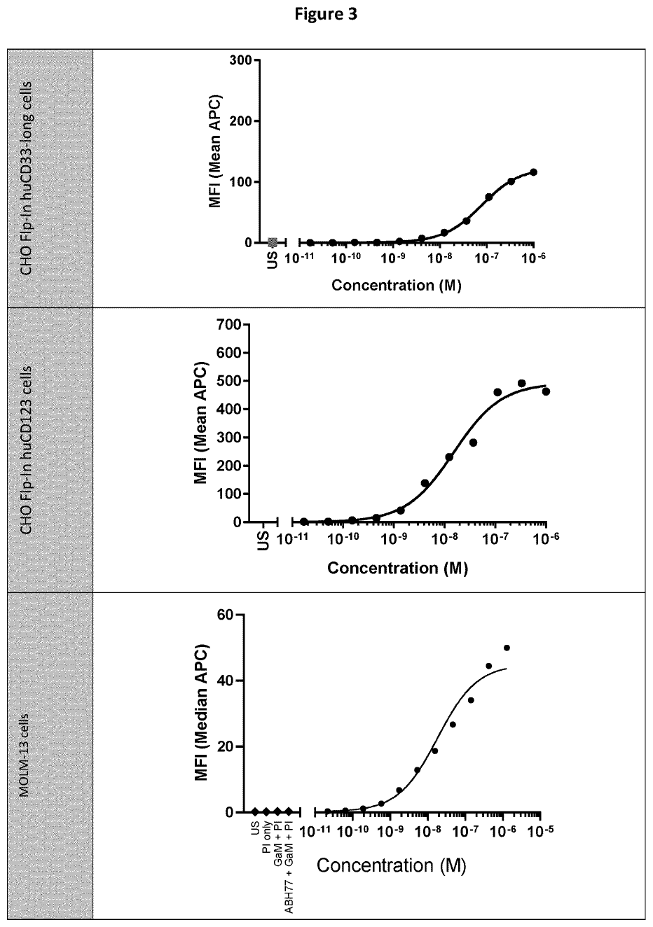

The binding of the CD123-CD33-TCR multispecific ISVD construct and the reference TCR-ISVD construct (consisting only of the TCRαβ building block (SEQ ID NO: 2) linked to the ALB23002 (SEQ ID NO: 5)) to human and cynomolgus T cells was evaluated in a competition setup using flow cytometry as described above (FIG. 3).

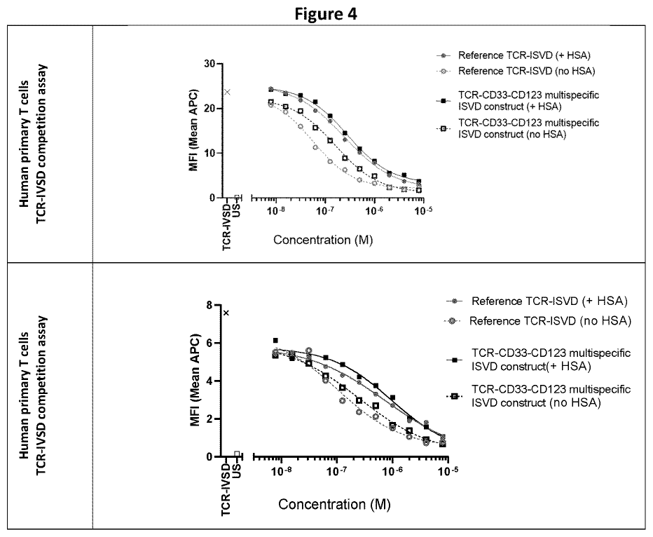

An exemplary example of the DRC is depicted in FIG. 4. The ISVD constructs were tested on multiple healthy donor T cells. Global IC₅₀ is shown in Table 8.

TABLE 8
Global IC₅₀ (M) of TCRαβ-CD33-CD123 multispecific ISVD
construct and reference TCR-ISVD construct in the human and
cynomolgus T cell competition assay as determined in flow cytometry

| | Primary human T cells | Primary cynomolgus T cells | Fold difference cyno |
|---|---|---|---|
| ISVD | Global IC50* | Global IC50* | versus human |
| TCRαβ-CD33- | 218 nM [190 nM-249 nM] | 729 nM [545 nM-976 nM] | 3.3 |
| CD123 | n = 4 | n = 7 | |
| (SEQ ID NO: 1) | | | |
| reference TCR- | 187 nM [134 nM-261 nM] | 493 nM [314 nM-772 nM] | 2.6 |
| ISVD | n = 4 | n = 7 | |

*global IC₅₀ (weighted)

In summary, the cross-reactivity of TCRαβ-CD33-CD123 multispecific ISVD construct towards cynomolgus primary T cells was confirmed. The EC₅₀ (=729 nM) on primary cynomolgus T cells was ~3-fold higher than on the human primary T-cells (EC₅₀ (=218M)

### 6.4 Example 4: Multispecific ISVD Construct Induced T Cell Mediated Target Cell Killing

### 6.4.1 Impedance-Based Cytotoxicity Assay

ISVD constructs were characterized for redirected T cell mediated killing in an impedance-based cytotoxicity assay (e.g. as described in WO2018091606A1) using human or cynomolgus primary effector T cells and adherent target cells. Changes in impedance induced by the adherence of target cells to the surface of an electrode were measured using the xCELLigence instrument (Roche). T cells are non-adherent and therefore do not impact the impedance measurements. The xCELLigence® RTCA MP instrument quantifies the changes in electrical impedance, displaying them as a dimensionless parameter termed cell index, which is directly proportional to the total area of tissue-culture well that is covered by cells. To each well of a 96 E-plate (ACEA Biosciences; 05 232 368 001) 50 μL of a 4× concentration HSA solution (in some assays 200 μM was used to have a final concentration of 50 μM, in other assays 120 μM was used to have a final concentration of 30 μM) in assay medium (target cell growth medium (without selection antibiotics)+1% penicillin/streptomycin (Life technologies Cat #15140) was added. Outer wells were not used and were filled with 200 μl medium or D-PBS. The 96 E-plate was placed in the xCELLigence® station (in the 37° C. incubator at 5% CO2) and a single measurement was performed to measure background impedance of the assay medium, in absence of cells. Subsequently, 50 μl target cells (2×10⁴ cells/well) in assay medium were seeded onto the 96 E-plate, and 50 μL of serially diluted ISVD construct solutions (4× concentration) in assay medium was added. (Final volume=200 L). After 30 min at room temperature, 50 μl of primary T cells (3×105 cells/well) in assay medium were added per well to achieve an effector to target ratio of 15:1. The plate was placed in the xCELLigence® station and impedance was measured every 15 min for 4 days. The data was analyzed at a fixed time point indicated in the results.

### 6.4.2 Flow Cytometry-Based Cytotoxicity Assay

ISVD constructs were characterized for redirected T cell mediated killing in a flow cytometry-based cytotoxicity assay using human or cynomolgus primary T cells as effector cells and non-adherent target cells. Target cells were labelled with 4 μM PKH26 membrane dye using the PKH26 red fluorescent cell linker kit (Sigma, PKH26GL-1KT) according to manufacturer's instruction. Effector cells (2.5×10⁵ cells/well) and PKH-labelled target cells (2.5×10⁴ cells/well) were co-incubated in 96-well V-bottom plates (Greiner Bio-one, #651 180) (effector versus target ratio of 10:1) in assay medium of the target cell line (target growth medium with 1% Penicillin/streptomycin (Life Technologies, 15140) and 50 μM Alburex HSA (CSL Behring, 2160-679)). For analysis of concentration-dependent cell lysis, serial dilutions of ISVD constructs in target assay medium were added to the cells and incubated for 18 h in a 5% CO₂ atmosphere at 37° C. After incubation, cells were pelleted by centrifugation and washed with FACS buffer (D-PBS (Gibco, 14190) with 10% FBS (Sigma, F7524) and 0.05% sodium azide (Acros organics, 19038)). Subsequently, cells were resuspended in 100 μL FACS buffer supplemented with 5 nM TO-PRO®-3 Iodide (642 [symbol] 661) (ThermoFisher Scientific, T3605) to distinguish live from dead cells. Cells were analyzed using a MACSQuant X flow cytometer (Miltenyi Biotec). Per sample, a total sample volume of 70 μl was acquired. Gating was set on PKH26 positive cells, and within this population, the TO-PRO®-3 positive cells were determined. The percent specific lysis=((% TO-PRO-3+no construct−% TO-PRO-3+with construct)/% TO-PRO-3+no construct))×100.

Functional cross-reactivity analysis of TCRαβ-CD33-CD123 multispecific ISVD construct towards CD33, CD123 and TCR was determined in the impedance-based cytotoxicity assay (xCELLigence) using human or cynomolgus primary T cells and adherent human or cynomolgus transfected CD33 or CD123 cells.

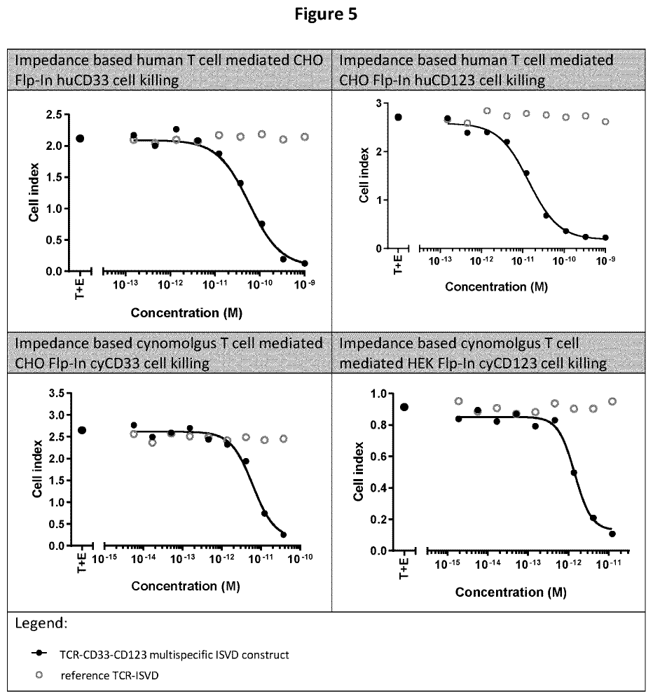

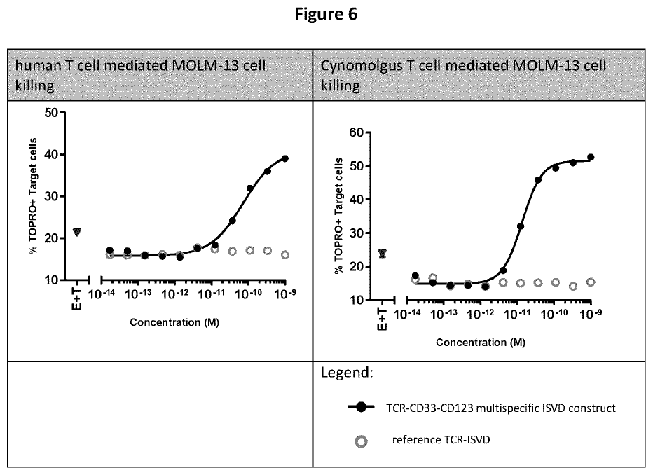

All assays, as described above in 6.4.1 and 6.4.2, were run in the presence of an excess amount of HSA, for the NANOBODY® ISVD to be fully saturated with HSA as described above. The reference TCR-ISVD was used as negative control. The results are shown in FIGS. 5 and 6 and Table 9.

### 6.4.3 Results

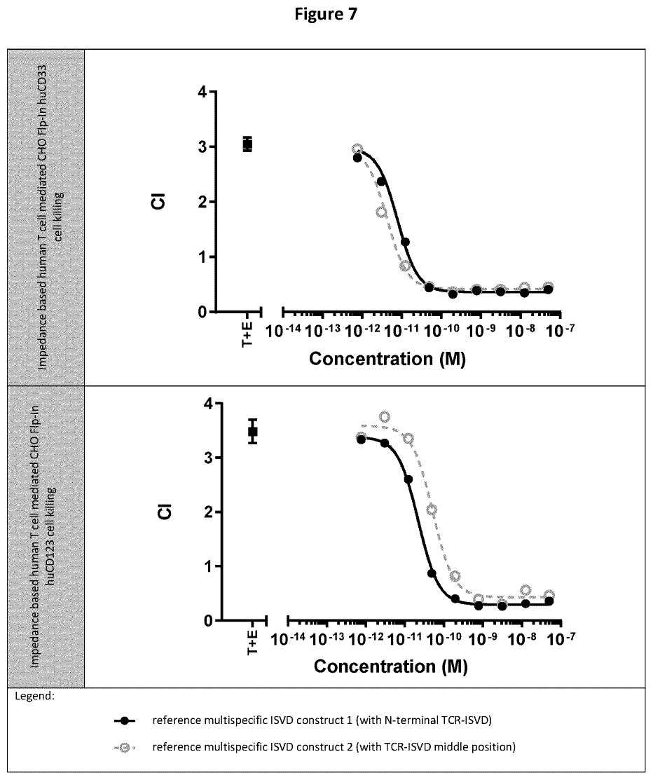

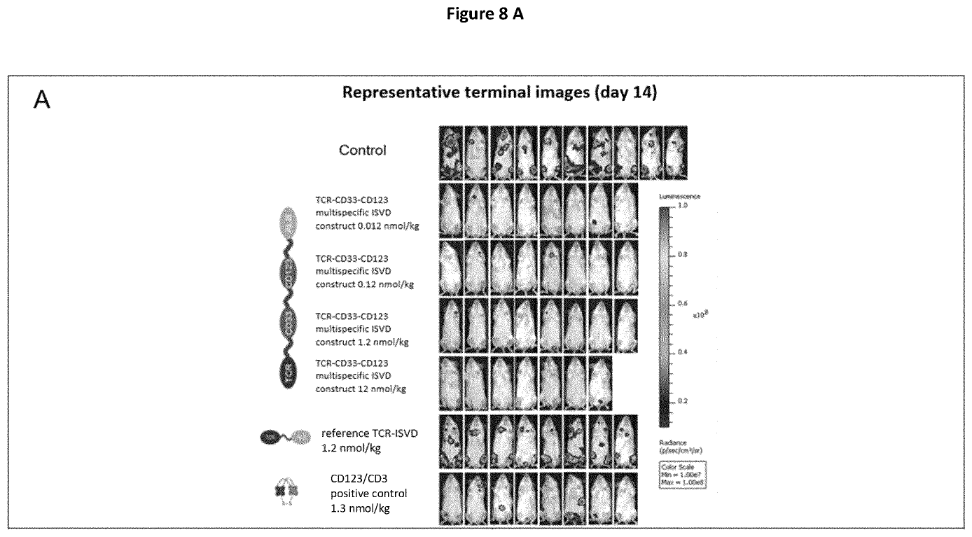

The assay results comparing human versus cynomolgus primary T cells are graphically illustrated in FIGS. 5 and 7. The evaluation of human T cell mediated cell killing of human CD33 and human CD123 transfected cells was performed using T cells from different human donors, which allowed to calculate the global IC₅₀ values (Table 9). The evaluation of cynomolgus T cell mediated killing of human CD33 and human CD123 transfected cells was performed using T cells from 1 cynomolgus monkey. The IC₅₀ values are summarized in Table 10.

TABLE 9
Global IC50 (M) of TCRαβ-CD33-CD123 in the impedance-based (xCELLigence) human T
cell mediated CD33+ or CD123+ cell killing assay using an effector to target ratio of 15 to 1
in thep resence of 50 μM HSA.

| | Impedance based human T cell | | | | Impedance based human T cell | | | |
|---|---|---|---|---|---|---|---|---|
| | mediated CHO hu CD33 cell killing | | | | mediated CHO hu CD123 cell killing | | | |
| | | Global | IC50 | IC50 | | Global | IC50 | IC50 |
| | | IC50 | (M) 95% | (M) 95% | | IC50 | (M) 95% | (M) 95% |
| ISVD | n | (M) | LCI | UCI | n | (M) | LCI | UCI |
| TCRαβ- | 4 | 1.1E−10 | 5.4E−11 | 2.4E−10 | 7 | 4.4E−11 | 2.5E−11 | 8.0E−11 |
| CD33-CD123 | | | | | | | | |
| (SEQ ID NO: 1) | | | | | | | | |

Global IC₅₀'s towards human target expressing cells were 5.4·10⁻¹¹M and 2.5·10⁻¹¹M respectively for CD33 and CD123.

TABLE 10
IC50 (M) of TCRαβ-CD33-CD123 in the impedance-based (xCELLigence) cynomolgus T
cell mediated CD33+ or CD123+ cell killing assay using an effector to target ratio of 15 to 1
in the presence of 50 μM HSA.

| | Impedance based cynomolgus T | | | Impedance based cynomolgus T | | |
|---|---|---|---|---|---|---|
| | cell mediated CHO Flp-In | | | cell mediated CHO Flp-In | | |
| | cynoCD33 cell killing | | | cynoCD123 cell killing | | |
| | | IC50 | IC50 | | IC50 | IC50 |
| | IC50 | (M) 95% | (M) 95% | IC50 | (M) 95% | (M) 95% |
| ISVD | (M) | LCI | UCI | (M) | LCI | UCI |
| TCRαβ- | 6.2E−12 | 5.1E−12 | 7.4E−12 | 1.4E−12 | 1.2E−12 | 1.7E−12 |
| CD33-CD123 | | | | | | |
| (SEQ ID NO: 1) | | | | | | |

To confirm the human cynomolgus cross-reactivity of TCRαβ-CD33-CD123 ISVD construct towards TCR, the ISVD construct was evaluated in a flow cytometry-based T cell mediated MOLM-13 cell killing using either human or primary T cells in combination with the CD33, CD123 double expressing human MOLM-13 target cell line in the presence of 50 μM HSA as described above. Graphical illustration of these results is shown in FIG. 6. Additional data of the TCRαβ-CD33-CD123 ISVD construct tested in the human T cell mediated killing assay using different donors were available, which allowed to calculate global EC₅₀ values (Table 11). The EC₅₀ values for the cynomolgus T cell mediated killing assays is shown in Table 12.

TABLE 11
Global EC₅₀ (M) of A025001562 (TCR-CD33-CD123 multispecific
ISVD construct, SEQ ID NO.: 1) in the flow cytometry human
T cell mediated MOLM-13 cell killing assay using an effector
to target ratio of 10 to 1 in the presence of 50 μM HSA.

| | Impedance based human T cell mediated | | | |
|---|---|---|---|---|
| | MOLM-13 cell killing | | | |
| | | Global EC₅₀ | EC₅₀ (M) | EC₅₀ (M) |
| ISVD | n | (M) | 95% LCI | 95% UCI |
| TCRαβ-CD33-CD123 | 13 | 1.80E−11 | 1.16E−11 | 2.78E−11 |
| (SEQ ID NO: 1) | | | | |

TABLE 12
EC50 (M) for TCRαβ-CD33-CD123 ISVD construct
in the cynomolgus T cell mediated MOLM-13 cell killing
assay using an effector to target ratio of 10 to 1.

Cynomolgus T cell mediated M
OLM-13 cell killing

| ISVD | EC₅₀ (M) | 95% LCI | 95% UCI |
|---|---|---|---|
| TCRαβ-CD33-CD123 | 1.36E−11 | 1.15E−11 | 1.62E−11 |
| (SEQ ID NO: 1) | | | |

In conclusion, TCRαβ-CD33-CD123 ISVD construct was functional both in the human and cynomolgus human target cell mediated killing assay. The global killing potency of the described ISVD towards the CD33/CD123 double positive AML cell line was 1.8·10⁻¹¹ M.

### 6.5 Example 5: Preclinical In Vivo-Efficacy of TCRαβ-CD33-CD123 Multispecific ISVD Construct in CD123⁺ Molm-13-Luc Disseminated AML Model Implanted in T Cell Humanized NSG Mice

### 6.5.1 Materials and Methods

### Cell Line and Human Material

The human AML-derived cell lines expressing CD123 Molm-13 was obtained from Deutsche Sammlung von Mikroorganismen und Zellkulturen (Braunschweig, Germany). Molm-13 cells were grown in culture (37° C., 5% CO₂, 95% humidity) in RPMI1640 Glutamax medium (completed with foetal cow serum 20%). Cells were infected with a luciferase vector (SV40-PGL4-Puro) carried by a non-replicative lentivirus; polyclonal Molm-13-luc were selected using 2 μg/ml puromycin.

### Human T-Cell Purification and Amplification for In Vivo Administration

Fresh human peripheral blood from healthy donors was supplied by EFS (Etablissement Français du Sang, Île-de-France, France.

Fresh human peripheral blood mononuclear cells (PBMCs) were isolated by Ficoll gradient centrifugation at 200 g for 40 min at room temperature without brake. The pellet was washed and resuspended in a final volume of 50 ml completed by phosphate-buffered saline (PBS). The total viable PBMCs number was defined by Vi-CELL counter (Beckman Coulter Life Sciences, Brea, CA, USA). The pellet was recovered in autoMACS Running Buffer (Miltenyi Biotec). T cells were isolated from PBMCs using the Pan T Cell Isolation Kit (Miltenyi Biotec) and autoMACS according to manufacturer instructions. Purified T cells were activated and expanded in vitro over 14 days using the T Cell TRANSACT matrix Activation/Expansion Kit (Miltenyi Biotec) based on CD3 and CD28 co stimulation. According with Miltenyi procedure, the activation protocol involved culturing T cells for 2 weeks in presence of TRANSACT matrix in TexMACS medium (Miltenyi Biotec), supplemented with 20 000 IU of soluble IL-2 and 1% penicillin-streptomycin (Gibco). At day 14 of expansion, T cells were harvested and resuspended in PBS at a final concentration of 5×10⁷ cells/ml, with 10⁷ cells administered through intraperitoneal (IP) injection to each animal. T cell viability prior to animal injection being tested to be higher than 85%.

### In Vivo Characterization

All in vivo experiments were approved by the Sanofi Ethical Committee and conducted in accordance with local and institutional Laws, Ethics and guidance in AAALAC accredited facilities.

TCRαβ-CD33-CD123 multispecific ISVD construct (SEQ ID NO:1) anti-tumor activity was evaluated after Molm13-luc AML engraftment in non-irradiated NOD.Cg-Prkdc^{scid} Il2rg^{tm1Wjl}/SzJ (NSG) mice (Charles River Laboratories, Saint-Germain-Nuelles, France). Female animals aged 6-8 weeks were implanted intravenously (IV) with 10⁶/0.2 ml Molm-13-luc cells per mouse on day 0. Same animals were intraperitoneally implanted with 10⁷/0.2 ml human T cells per mouse on day 1.

Animals were distributed among groups based on all-body bioluminescence imaging (BLI) signal homogeneity and tumor myeloid engraftment evaluated by long bones signal segmentation on day 3. Mice were treated IV with TCRαβ-CD33-CD123 multispecific ISVD construct (SEQ ID NO:1) at 12, 1.2, 0.12, and 0.012 nmol/kg Q2D from day 4 to day 12 or with a reference, not having a CD33 or CD123 binding ISVD, (TCRαβ-ISVD (SEQ ID NO: 2) linked to Alb-ISVD (SEQ ID NO:5)) at 1.2 nmol/kg QD (days 4-13) or CD123/CD3 positive control 1.3 nmol/kg QD (days 4-13), see Table 13. Longitudinal in vivo bioluminescence imaging (BLI) was performed to monitor disseminated tumor growth. Mice were sacrificed on day 14 and autopsied under BLI for impact evaluation in deep soft tissues such as liver, spleen, ovaries, and abdominal fat.

TABLE 13
Compound evaluation study design

| | Dose | Volume/ | | Animal |
|---|---|---|---|---|
| Treatment Group | (nmol/Kg) | inj Route | Schedule | number |
| Control | — | — | — | 10 |
| TCR-CD33-CD123 | 12 | 0.2 ml IV | Q2dx5 | 7^{a} |
| (SEQ ID NO: 1) | | | (4, 6, 8, 10, 12) | |
| | 1.2 | 0.2 ml IV | Q2dx5 | 8 |
| | | | (4, 6, 8, 10, 12) | |
| | 0.12 | 0.2 ml IV | Q2dx5 | 8 |
| | | | (4, 6, 8, 10, 12) | |
| | 0.012 | 0.2 ml IV | Q2dx5 | 8 |
| | | | (4, 6, 8, 10, 12) | |
| TCR ISVD construct | 1.2 | 0.2 ml IV | Qdx10 (4-13) | 8 |
| positive control | 1.3 | 0.2 ml IV | Qdx10 (4-13) | 8 |

^{a}one non-responding animal due to lack of T cell engraftment was not include in analysis

### Data Collection and Efficacy Criteria

Animal body weight was monitored from day 3 to the end of assay to follow impact of therapy. A dosage producing a 20% weight loss or 15% weight loss for 3 consecutive day or 10% or more drug deaths, was considered an excessively toxic dosage. Animal body weights included the tumor weights.

Tumor growth was assessed by in vivo BLI using the IVIS Lumina XRMS imager (PerkinElmer, Waltham, MA, USA) with the Living Image 4.5.2 acquisition software (PerkinElmer) through luciferase activity measurement in vivo on days 3, 7, 10, and 14 post-tumor injections, using beetle luciferin potassium salt 160 mg/kg Ip injection 15 minutes before image processing of animals anesthetized with Kétamine®/Xylazine® (120 mg/kg; 6 mg/kg IM, 5 ml/kg) 5 minutes before image. Tumor growth was based on bioluminescence signal curves (expressed in photons/second).

Tumor growth was followed in all body and long bones in posteriors legs by BLI signal measurements at days 7, 10 and 14 after tumor implantation. The primary efficacy end points are ratio of change in tumor signal changes from baseline between treated and control groups (T/C), partial regression (PR) and complete regression (CR).

Tumor growth based on bioluminescence signal curves (expressed in Phot/sec) in time was drawn for each animal of each treatment group and represented as median curve±MAD, both for all body (linear scale) and bone segmented signals (Log scale). Changes in tumor bioluminescence signal for each treated (T) and control (C) are calculated for each animal and each day by subtracting the tumor signal on the day of first treatment (staging day) from the tumor signal on the specified observation day. The median T is calculated for the treated group and the median C is calculated for the control group. Then the ratio T/C is calculated and expressed as a percentage:

dT/dC=[(median T end−median T day 3)/(median C end−median C day 3)]×100

The dose is considered as therapeutically active when dT/dC is lower than 42% and very active when dT/dC is lower than 10%. If dT/dC is lower than 0, the dose is considered as highly active and the percentage of regression is dated:

Percent tumor regression is defined as the % of tumor signal decrease in the treated group at a specified observation day compared to its signal on the first day of treatment.

For each animal, % regression is calculated at specific time-points. Considering the risk of signal variability due to luciferin kinetics variability with ip miss injection, true regressions are considered when they are observed at least at two consecutive time points for each animal.

$$\% regression ( at t ) = \frac{{Signal}_{t 0} - {Signal}_{t}}{{Signal}_{t 0}} \times 100$$

Partial regression (PR): Regressions are defined as partial if the tumor Signal decreases bellow the tumor Signal at the start of treatment at two consecutive time points, one being below 50% of start signal. Complete regression (CR): Regressions are defined as Complete if the tumor Signal decreases below 80% of start signal.

### Biostatistical Analysis

Tumor growth based on bioluminescence signal curves over time was measured for each animal of each treatment group. For longitudinal in vivo BLI data, a two non-parametric two-way analysis of variance-Type (ANOVA-Type), followed by two contrast analyses with Bonferroni-Holm adjustment for multiplicity were performed with repeated measures by day: p>0.05: NS, 0.05<p>0.01: *, p<0.01: **. For terminal ex vivo BLI data, a one-way ANOVA with factor group on rank-transformed bioluminescence signal was performed. Descriptive statistics with medians±median absolute deviation was provided by group and day of measurement: p>0.05: NS, 0.05<p>0.01: *, p<0.01:

### 6.5.2 Results

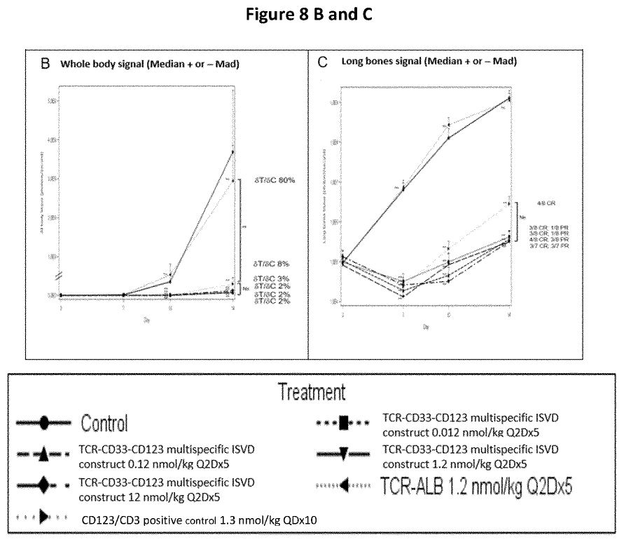

TCRαβ-CD33-CD123 multispecific ISVD construct induces anti-leukemic effects in Molm-13-luc AML xenograft model in vivo (FIG. 8A).

TCRαβ-CD33-CD123 multispecific ISVD construct administered intravenously every 2 days in the Molm-13-luc xenograft model in the presence of human effector T cells (T cell/Tumor ratio R=10) was well tolerated at all doses. No evidence of adverse events or alterations of body weight were observed under treatment. TCRαβ-CD33-CD123 multispecific ISVD construct inhibited whole body tumor growth at all tested doses with same activity (dT/dC of 2% (p<0.0001) Vs 2% (p<0.0001), 2% (p<0.0001) and 3% (p<0.0001) at 0.012; 0.12; 12 and 12 nmol/kg respectively (FIG. 8A, 8B).

A sum of the longest diameter (LD) for all target lesions was the baseline sum LD. The baseline sum LD was used as reference by which to characterize the objective tumor response.

In long bones, a 3/8 Complete Response (CR; disappearance of all lesions) and a 1/8 Partial Response (PR; at least a 30% decrease in the sum of the LD of target lesions, taking as reference the baseline sum LD) were observed at 0.012 nmol/kg.

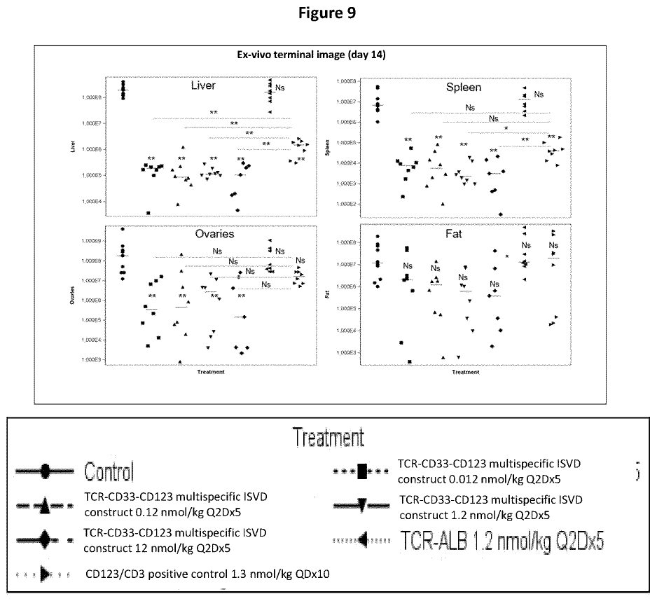

A 3/8CR and 1/8PR were observed at 0.12 nmol/kg, a 4/8CR and 3/8PR were observed at 1.2 nmol/kg, and a 3/7CR and 3/7PR were observed at 12 nmol/kg (FIG. 8C). Reference TCR-ISVD construct was overall inactive on Molm13-luc tumor growth at 1.2 nmol/kg (dT/dC of 80%). CD123/CD3 positive control 1.3 nmol/kg inhibited tumor growth with dT/dC 8% (NS compared to A025001562 (TCR-CD33-CD123 multispecific ISVD construct, SEQ ID NO.: 1) treatments) associated with 4/8 CR in long bones (FIG. 8A, 8B). Based on terminal ex vivo bioluminescence imaging, TCRαβ-CD33-CD123 multispecific ISVD construct significantly inhibited tumor growth in liver (p<0.0001), spleen (p<0.0001) and ovaries (p<0.0001) but not in abdominal fat at all tested doses, when reference ISVD TCR-HLE was inactive in all tissues, and CD123/CD3 positive control inhibited significantly the tumor load in liver (p<0.0001) and spleen (p<0.0001) but not in ovaries (NS) or abdominal fat tissues. (FIG. 9)

### 6.6 Example 6: T Cell Mediated Human Target Cell Killing

### 6.6.1 Materials and Methods

The ISVD constructs were characterized for redirected T cell mediated killing in a flow cytometry-based cytotoxicity assay using human primary T cells as effector cells and non-adherent target cells. CD123 and/or CD33 positive target cells (MOLM-13, DSMZ ACC 554, U-937, ATCC® CRL1593.2, and KG-1a, ATCC® CCL246.1™) were labelled with 4 μM PKH-26 membrane dye using the PKH26 red fluorescent cell linker kit (Sigma, PKH26GL-1KT) according to manufacturer's instructions. Effector cells (2.5×10⁵ cells/well) and PKH-labelled target cells (2.5×10⁴ cells/well) were co-incubated in 96-well V-bottom plates (Greiner Bio-one, #651 180) (effector versus target ratio of 10:1) in assay medium of the target cell line (target growth medium without antibiotics). For analysis of concentration-dependent cell lysis, serial dilutions of compounds in target assay medium were added to the cells and incubated for 18 h in a 5% CO₂ atmosphere at 37° C. After incubation, cells were pelleted by centrifugation and washed with FACS buffer (D-PBS from Gibco, with 10% FBS from Sigma and 0.05% sodium azide from Merck). Subsequently, cells were resuspended in FACS buffer supplemented with 5 nM TO-PRO®-3 Iodide (642/661) (ThermoFisher Scientific, T3605) to distinguish live from dead cells. Cells were analyzed using a FACS Array flow cytometer (BD Biosciences). Per sample, a total sample volume of 80 μL was acquired. Gating was set on PKH26 positive cells, and within this population, the TO-PRO®-3 positive cells were determined. Percent specific lysis=((% TO-PRO®-3+_{no ISVD}−% TO-PRO®-3+_{with ISVD})/(% TO-PRO®-3+_{no ISVD}))×100.

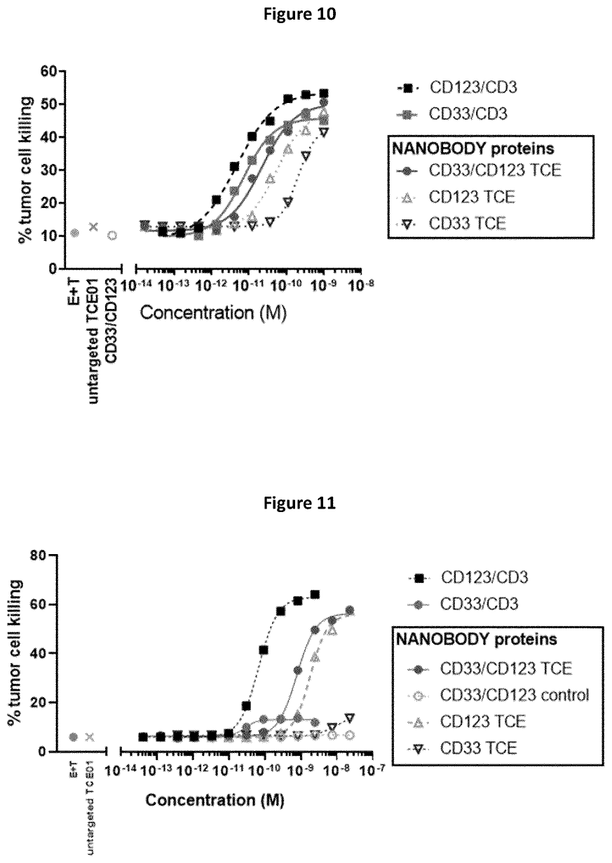

Multispecific TCRαβ-CD33-CD123 ISVD construct according to the invention was compared to corresponding constructs, where either the CD33 binding ISVD or the CD123 binding ISVD was replaced by an irrelevant ISVD IRR (not binding to CD33 and not binding to CD123; Table 23) as well as a CD123/CD3 positive control and a CD33/CD3 positive control. Results are shown in FIGS. 10, 11 and 12.

### 6.6.2 Results

As can be seen in FIG. 10, in the MOLM-13 cells all tested compounds triggered tumor cell killing, which is to be expected since MOLM-13 cells are both CD123 and CD33 positive. Of the ISVD constructs, the dual-targeting format (CD33/CD123 TCE) had the most potent tumor cell killing.

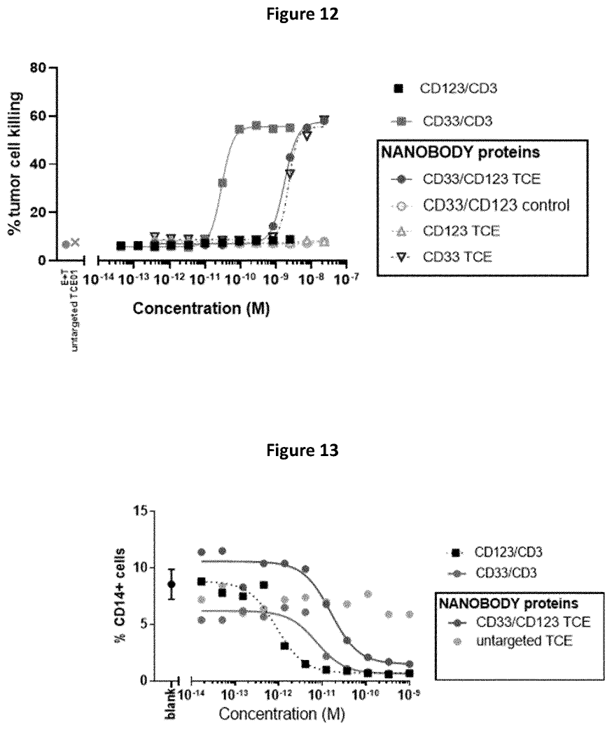

Killing potencies and percentages of lysis in the U-937 and KG-1a cells are depicted in Table 24 as well as FIGS. 11 and 12.

TABLE 23
Sample ID and the composition of the NANOBODY ® ISVDs
and linkers

| Sample | Target | | Target | | Target |
|---|---|---|---|---|---|
| ID | ISVD1 | Linker | ISVD2 | Linker | ISVD3 |
| A | TCR | 9GS | CD33 | 9GS | CD123 |
| B | TCR | 9GS | CD33 | 9GS | IRR |
| C | TCR | 9GS | IRR | 9GS | CD123 |

TABLE 24
EC50 (M) and % lysis for the trivalent NANOBODY® ISVDs in the human T cell
mediated U-937 or KG-1a cells cell killing assay using an effector to target ratio of 10 to

| | Flow cytometry based human T cell | | | | Flow cytometry based human T cell | | | |
|---|---|---|---|---|---|---|---|---|
| | mediated U-937 cell killing | | | | mediated KG-1a cell killing | | | |
| | EC50 | EC50 (M) | EC 50 (M) | % | EC50 | EC 50 (M) | EC 50 (M) | % |
| sample ID | (M) | 95% LCI | 95% UCI | Lysis | (M) | 95% LCI | 95% UCI | Lysis |
| CD33/CD3 | 1.1E−11 | 9.8E−12 | 1.3E−11 | 55 | 8.7E−12 | 6.9E−12 | 1.1E−11 | 15 |
| CD123/CD3 | 8.9E−13 | 7.0E−13 | 1.1E−12 | 13 | 3.0E−12 | 2.4E−12 | 4.0E−12 | 58 |
| A | 5.9E−11 | 4.6E−11 | 7.9E−11 | 57 | 3.6E−11 | 2.8E−11 | 4.9E−11 | 54 |
| B | 4.7E−11 | 4.2E−11 | 5.2E−11 | 55 | 3.0E−10 | 2.3E−10 | 4.2E−10 | 30 |
| C | ~4.4E−12 | / | / | 17 | 3.8E−11 | 2.9E−11 | 5.1E−11 | 64 |

The TCR-CD123 single targeting ISVD hardly induced killing of CD123-U-937 cells, as was also observed for the CD123/CD3 positive control. Similarly, the CD33 single targeting ISVD induced only low level killing of CD33−/+KG-1a cells, as was also observed for the CD33/CD3 positive control. The dual-targeting TCRαβ-CD33-CD123 ISVD (construct A) on the other hand, demonstrated potent tumor cell killing on both CD33− and CD123− cell lines, thus exemplifying the advantage of the dual-targeting approach.

### 6.7 Example 7: Cytokine Release Assay

As Cytokine Release Syndrome (CRS) is a known side effect of T cell engagers, the cytokine release profile of the TCRαβ-CD33-CD123 multispecific ISVD construct according to the invention was determined in an autologous healthy donor PBMC assay. In parallel, autologous depletion of monocytes within human PBMCs was evaluated. The functionality of the ISVD according to the invention was compared with 2 tool molecules: a CD123/CD3 positive control and a CD33/CD3 positive control. An untargeted T cell engager (TCE) ISVD was taken along as negative control.

### 6.7.1 Materials and Methods

A total of 200 000 PBMCs (100 μL @ 2×106 cells/mL in culture medium), isolated from whole blood using Leucosep™ tube containing Lymphoprep™ solution, were transferred to 96-well V-bottom clear well plates (Greiner CELLSTAR® 96 well plates; 651 180).

Next, 50 μl of the serial dilution of the compounds to be tested or 50 μL culture medium for the empty wells were added to the wells.

Culture plates containing treated PBMCs were incubated at 37° C. at 5% CO2 for 20 h. After overnight (20h) incubation, PBMCs were centrifuged at 300 g for 2 min. The supernatant was collected and transferred to a new 96 well storage plate, frozen at −20° C. for cytokine measurements. The cell pellets were suspended in 100 μL cold FACS buffer and washed 1 time with 100 μL FACS buffer.

After that, PBMCs were centrifuged at 300 g for 2 min at 4° C. Supernatant was discarded and cells were resuspended in 30 μL diluted Fc block (1/200 in FACS buffer) (BD, 564220) and incubated for 10 minutes at room temperature.

Next, 30 μL of (2×) antibody staining mix (CD123, HLA-DR and CD14 antibodies) was added to the PBMC suspensions and incubated 30 minutes at 4° C. in the dark.

Then, cell suspensions were washed 2 times and resuspended in 50 μL diluted TO-PRO™-3 Iodide. The plates were read on MACSQuantX. The read out was the detection of viable cell numbers for each subset by flow cytometry. Monocytes were quantified by SSC % CD14+ (FIG. 13).

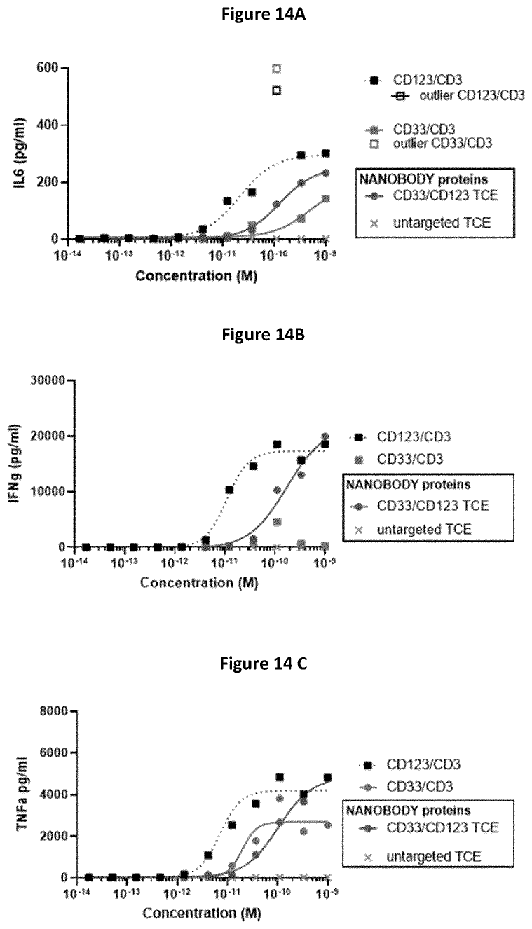

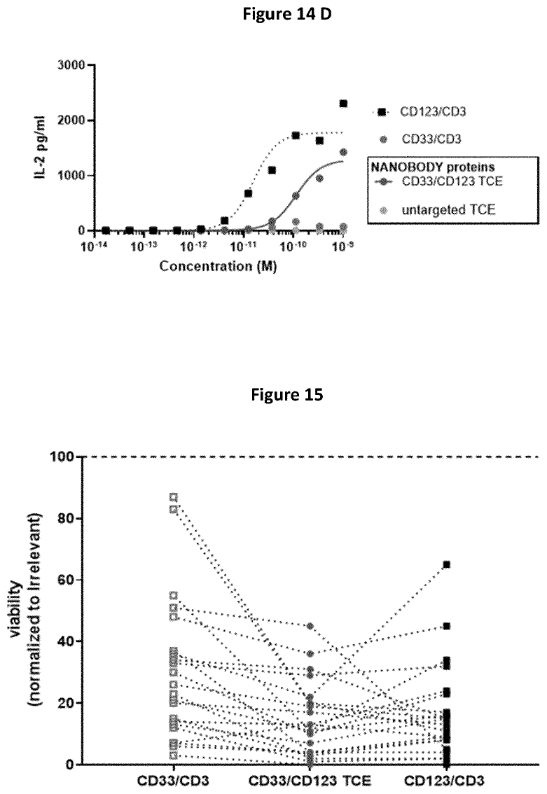

The frozen supernatant from the overnight incubation of the human PBMCs was used to measure a panel of cytokines including IL-2, IL-6, IFNγ, TNFα using a multiplex bead assay from Bio-rad. The assay was performed according to the manufacturer's guidelines. Data from the reaction were acquired using the Luminex FlexMAP 3D system (FIG. 14).

### 6.7.2 Results

The TCRαβ-CD33-CD123 multispecific ISVD construct induced depletion of monocytes associated with cytokine production in all donors. The untargeted TCE did not show any killing nor cytokine release (see FIG. 13). The data shown in FIG. 13 is data from 1 donor, which is representative of the data of all 6 donors that provided PBMCs.

Additionally, both the potencies and the top levels of the cytokines induced by the ISVD construct according to the invention were substantially lower compared to the CD123/CD3 control compound. In comparison with the CD33/CD3 control, IL-6 and TNFα production induced by the ISVD construct according to the invention were comparable. Although the level of IL-2 and IFNγ were higher than with the CD33/CD3 control, the levels were still acceptable.

Based on this it can be concluded that the ISVD construct according to the invention does not have a higher risk to induce CRS than other CD123 or CD33 targeting compounds and should be safe for use in humans.

### 6.8 Example 8: Ex Vivo Primary AML Cell Killing Assay

### 6.8.1 Materials and Methods

Lysis of AML blasts mediated by CD33/CD3 positive control was tested as follows: AML samples from patients with a primary diagnosis or relapse were used in an ex vivo co-culture system without the addition of any other cells. Hence, the E:T ratio was determined by the number of residual T cells within the primary AML sample. Whole blood samples from AML patients were provided by public hospitals in Marseille (La Conception, AP-HM) or Montpellier or a central lab.

To start with, 1 mL of whole blood samples were distributed per 50 mL tubes and 40 ml of (1×) red blood cell lysis buffer was added and incubated for 10 minutes at room temperature. After the incubation, the pellets were washed with PBS (Eurobio CS1PBS01-01) and resuspended in 1 ml of culture media ((RPMI (Eurobio cat #CM1 RPM00-01) FCS 10% (Sigma cat #F2442-500 ml 17L484), non-essential amino acid 1% (Eurobio cat #CSTAAN00), Glutamine 1% (Eurobio cat #CSTGLU00) Sodium Pyruvate 1% (Eurobio cat #CSTVAT00), Penicillin/Streptomycin, (Eurobio cat #CABPES01)).

Next, 300 μL of AML blast cells were added to 700 μl of culture medium per well in a 6 well plate in the presence of 1 ml (2×) saturating concentration of TCRαβ-CD33-CD123 multispecific ISVD construct according to the invention (CD33/CD123 TCE), positive control (CD33/CD3 or CD123/CD3) or negative control (untargeted TCE) and incubated at 37° C. at 5% CO2. After 4 days of incubation, the cells were harvested and washed 1 time with PBS.

Next, Zombie Violet viability marker (diluted 1/100 in PBS) was added to the cell pellets and incubated for 5 minutes at room temperature in the dark.

Then, cells were washed and stained with an antibody cocktail (CD45, CD33, CD34, CD38, CD14, CD123, CD11 antibodies) and incubated 10 minutes on ice.

Next, cells were washed and fixed with 1.5 ml of PBS+2% paraformaldehyde for 30 minutes at 4° C. After the 30-minute incubation, the paraformaldehyde was diluted with 9 ml of diluent. The data were and analyzed on Cytoflex cytometer. Results are shown in FIGS. 15-17.

### 6.8.2 Results

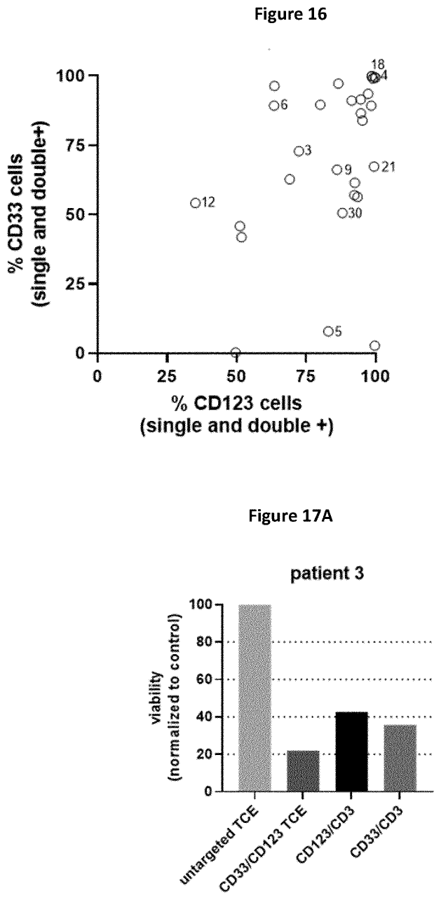

The percentage of CD33 or CD123 positive cells per AML sample are shown in FIG. 16. As can be seen, all patients had a high number of both CD33 and CD123 positive cells. Additionally, the AML patients had a wide range of disease subtypes. Therefore, the AML samples were suitable for use in this assay.

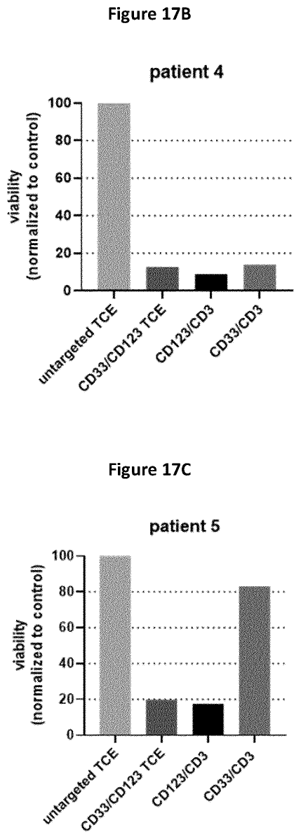

In FIG. 15, the AML blast cell killing in all AML patient samples is shown. Each dot represents the blast count surviving after treatment with either the ISVD according to the invention or one of the positive controls normalized to the negative control (untargeted TCE). The results of a few separate patients have been highlighted in FIG. 17.

As can be seen, the cell viability of the blast cells treated with the ISVD according to the invention was, on average, the lowest compared to the positive controls. Cells from all patients showed a clear response, although the response varied from patient to patient. This shows that the ISVD according to the invention is capable of targeted cell killing of CD123 and CD33 positive tumor cells.

### 6.9 Example 9: Nonhuman Primate (NHP) Study

The pharmacokinetic (PK), pharmacodynamic (PD) and nonclinical safety profile of the TCRαβ-CD33-CD123 multispecific ISVD construct according to the invention was evaluated in studies in cynomolgus monkeys.

### 6.9.1 Materials and Methods

The ISVD construct according to the invention was administered to a total of 4 males Cynomolgus monkeys as a single dose 1-hour continuous intravenous (IV) infusion followed by a 21-day observation period. The systemic exposure and potential toxicity of the ISVD was determined, and PD endpoints (immune cells evaluation) were evaluated. The study design is shown in Table 25.

TABLE 25
Study design: exploratory single dose
IV study in male cynomolgus monkeys

| | Dose | Number of | Sex and animal |
|---|---|---|---|
| Group | (μg/kg) | animals | identity number |
| 1 | 0.04 | 2 | M1, M2 |
| 2 | 0.4 | 2 | M3, M4 |

Abbreviations: M: male

### 6.9.2 Safety

The single 1-hour continuous IV infusion of the ISVD construct to Cynomolgus monkeys was well tolerated. No mortality and no treatment-related clinical signs occurred during the course of the study. No treatment-related body weight changes and body temperature changes were observed.

Cytokine evaluation showed only very minimal increases in IL-6 (peak occurring at 2 hours and/or 6 hours after the start of the infusion and returning to baseline within 24 hours) associated with the ISVD construct administered as a single-dose at 0.04 μg/kg. No changes in IFN-γ, IL-1β, IL-2, IL-8 and TNF-α levels were observed at 0.04 μg/kg. Very minimal to minimal increases in IL-2, IL-6, IFN-γ and TNF-α (peak occurring at 2 hours and/or 6 hours after the start of the infusion and returning to baseline within 24 hours) were associated with the ISVD construct administered as a single-dose at 0.4 μg/kg. No changes related to the TCR-CD33-CD123 multispecific ISVD construct according to the invention were observed on IL-1β and IL-8 levels on monkeys at 0.4 μg/kg.

Therefore, it can be concluded that the ISVD construct according to the invention was well tolerated in this NHP model.

### 6.9.3 Pharmacokinetics

After a 1-hour infusion of the ISVD construct, serum levels were quantifiable in monkeys up to 3 days at 0.04 μg/kg and up to 7 days at 0.4 μg/kg. Overall, a clearance of around 0.07 L/day/kg was observed after IV infusion at 0.04 μg/kg and 0.4 μg/kg dose levels. The terminal elimination half-life has been estimated to be close to 1 day. From 0.04 to 0.4 μg/kg, ISVD construct exposure (AUC) increased slightly less than expected by dose proportionality, with, for a 10-fold increase in dose, a 7.7-fold increase in exposure, likely due to the lower exposure observed in one monkey (M3). The PK parameters are reported in Table 26.

TABLE 26
TCRαβ-CD33-CD123 multispecific ISVD construct toxicokinetic parameters.

| Dose | Cₑₒᵢ | Tₘₐₓ | AUCₗₐₛₜ | Tₗₐₛₜ | AUC | CL | Vₛₛ | T_{1/2z} |
|---|---|---|---|---|---|---|---|---|
| (μg/kg) | (pg/mL) | (days) | (day*pg/mL) | (days) | (day*pg/mL) | (L/day/kg) | (L/kg) | (days) |
| 0.04 | 830 | 0.06 | 523 | 2.5 | 621 | 0.0664 | 0.0776 | 1.12 |
| | | [0.04- | | [2-3] | | | | |
| | | 0.08] | | | | | | |
| 0.4 | 7980 | 0.04 | 4590 | 5 | 4790 | 0.100 | 0.0867 | 1.37 |
| | | [0.04- | | [3-7] | | | | |
| | | 0.04] | | | | | | |

Abbreviations:
AUC: Area Under the Curve from 0 to infinity,
AUClast: area under plasma concentration versus time curve from 0 to the time of the last measurable concentration;
CL: clearance;
Ceol: plasma concentration at the end of infusion;
t1/2: terminal half-life;
tlast: last measurable concentration;
tmax: first time to reach Cmax;
Vss: volume of distribution at steady state

### 6.9.4 Pharmacodynamics

The single dose IV infusion of the ISVD construct to cynomolgus monkeys at 0.04 and 0.4 μg/kg induced changes in CD123+ cell populations and CD33+ monocytes at both dose levels. These changes consisted of:
a total decrease in total CD123+ cells number observed for all animals starting from 6 hours after the start of the infusion. This decrease was maintained for all animals throughout the study.
a total decrease in monocytes CD33+ cells number observed for all animals starting from 6 hours after the start of the infusion (and up to day 1 for animal M1 receiving the ISVD construct at 0.04 ug/kg). A full recovery was observed at Day 1 for animal M3.

Also, a total decrease in CD4+ and CD8+ cells numbers was observed for all animals starting from 6 hours after the start of the infusion. For CD8+ cells, a full recovery was observed at Day 3 with a rebound effect for animal M4. For CD4+ cells, a full recovery was observed on Day 3 for animal M4.

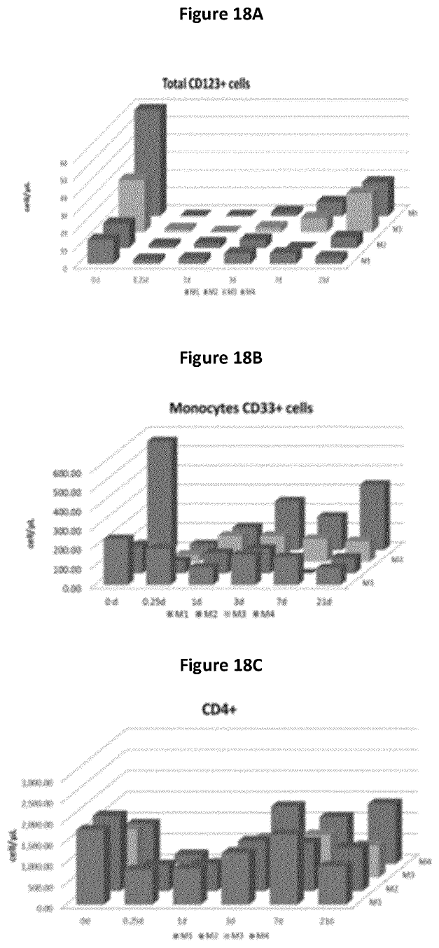

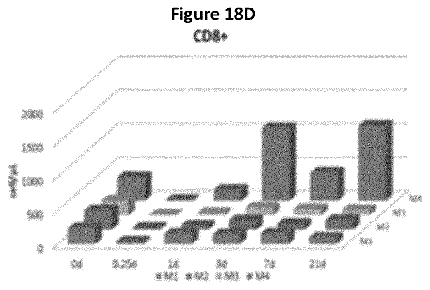

The cell counts have been visualized in FIG. 18.

### 7 INDUSTRIAL APPLICABILITY

The polypeptides, nucleic acid molecules encoding the same, vectors comprising the nucleic acids and compositions described herein may be used for example in the treatment of subjects suffering from acute myeloid leukemia.

1. A polypeptide that comprises or consists of at least three immunoglobulin single variable domains (ISVDs), optionally linked via one or more peptidic linkers, wherein each of said ISVDs comprises three complementarity determining regions (CDR1 to CDR3, respectively), and wherein:
a. a first ISVD is capable of binding to T cell receptor αβ (TCRαβ) and comprises:
i. a CDR1 having the amino acid sequence of SEQ ID NO: 6 or an amino acid sequence having 2 or 1 amino acid difference(s) with SEQ ID NO: 6;
ii. a CDR2 having the amino acid sequence of SEQ ID NO: 10 or an amino acid sequence having 2 or 1 amino acid difference(s) with SEQ ID NO: 10; and
iii. a CDR3 having the amino acid sequence of SEQ ID NO: 14 or an amino acid sequence having 2 or 1 amino acid difference(s) with SEQ ID NO: 14;
b. a second ISVD is capable of binding to CD33 and comprises:
iv. a CDR 1 having the amino acid sequence of SEQ ID NO: 7 or an amino acid sequence having 2 or 1 amino acid difference(s) with SEQ ID NO: 7;
v. a CDR2 having the amino acid sequence of SEQ ID NO: 11 or an amino acid sequence having 2 or 1 amino acid difference(s) with SEQ ID NO: 11; and
vi. a CDR3 having the amino acid sequence of SEQ ID NO: 15 or an amino acid sequence having 2 or 1 amino acid difference(s) with SEQ ID NO: 15; and
c. a third ISVD is capable of binding to CD123 and comprises:
vii. a CDR1 having the amino acid sequence of SEQ ID NO: 8 or an amino acid sequence having 2 or 1 amino acid difference(s) with SEQ ID NO: 8;
viii. a CDR2 having the amino acid sequence of SEQ ID NO: 12 or an amino acid sequence having 2 or 1 amino acid difference(s) with SEQ ID NO: 12; and
ix. a CDR3 having the amino acid sequence of SEQ ID NO: 16 or an amino acid sequence having 2 or 1 amino acid difference(s) with SEQ ID NO: 16,
wherein the order of the ISVDs indicates their relative position to each other considered from the N-terminus to the C-terminus of said polypeptide.

2. The polypeptide according to claim 1, wherein:
a. said first ISVD comprises a CDR1 having the amino acid sequence of SEQ ID NO: 6, a CDR2 having the amino acid sequence of SEQ ID NO: 10 and a CDR3 having the amino acid sequence of SEQ ID NO: 14;
b. said second ISVD comprises a CDR1 having the amino acid sequence of SEQ ID NO: 7, a CDR2 having the amino acid sequence of SEQ ID NO: 11 and a CDR3 having the amino acid sequence of SEQ ID NO: 15; and
c. said third ISVD comprises a CDR1 having the amino acid sequence of SEQ ID NO: 8, a CDR2 having the amino acid sequence of SEQ ID NO: 12 and a CDR3 having the amino acid sequence of SEQ ID NO: 16.

3. The polypeptide according to claim 1, wherein:
a. the amino acid sequence of said first ISVD comprises a sequence identity of more than 90% with SEQ ID NO: 2;
b. the amino acid sequence of said second ISVD comprises a sequence identity of more than 90% with SEQ ID NO: 3; and
c. the amino acid sequence of said third ISVD comprises a sequence identity of more than 90% identity with SEQ ID NO: 4.

4. The polypeptide according to claim 1, wherein:
a. said first ISVD comprises the amino acid sequence of SEQ ID NO: 2;
b. said second ISVD comprises the amino acid sequence of SEQ ID NO: 3; and
c. said third ISVD comprises the amino acid sequence of SEQ ID NO: 4.

5. The polypeptide according to claim 1, wherein said polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units.

6. The polypeptide according to claim 5, in which said one or more other groups, residues, moieties or binding units that provide the polypeptide with increased half-life is chosen from the group consisting of a polyethylene glycol molecule, serum proteins or fragments thereof, binding units that can bind to serum proteins, an Fc portion, and small proteins or peptides that can bind to serum proteins.

7. The polypeptide according to claim 5, in which said one or more other groups, residues, moieties or binding units that provide the polypeptide with increased half-life is chosen from the group consisting of binding units that can bind to serum albumin or a serum immunoglobulin.

8. The polypeptide according to claim 7, in which said binding unit that provides the polypeptide with increased half-life is a fourth ISVD that is capable of binding to human serum albumin.

9. The polypeptide according to claim 8, wherein the fourth ISVD that is capable of binding to human serum albumin comprises:
i. a CDR1 having the amino acid sequence of SEQ ID NO: 9 or an amino acid sequence having 2 or 1 amino acid difference(s) with SEQ ID NO: 9;
ii. a CDR2 having the amino acid sequence of SEQ ID NO: 13 or an amino acid sequence having 2 or 1 amino acid difference(s) with SEQ ID NO: 13; and
iii. a CDR3 having the amino acid sequence of SEQ ID NO: 17 or an amino acid sequence having 2 or 1 amino acid difference(s) with SEQ ID NO: 17.

10. The polypeptide according to claim 8, wherein the ISVD that is capable of binding to human serum albumin comprises a CDR1 having the amino acid sequence of SEQ ID NO: 9, a CDR2 having the amino acid sequence of SEQ ID NO: 13 and a CDR3 having the amino acid sequence of SEQ ID NO: 17.

11. The polypeptide according to claim 8, wherein the amino acid sequence of said ISVD that is capable of binding to human serum albumin comprises a sequence identity of more than 90% with SEQ ID NO: 5.

12. The polypeptide according to claim 8, wherein said ISVD that is capable of binding to human serum albumin comprises the amino acid sequence of SEQ ID NO: 5.

13. The polypeptide according to claim 1, wherein the amino acid sequence of the polypeptide comprises a sequence identity of more than 90% with SEQ ID NO: 1, optionally wherein the polypeptide consists of the amino acid sequence of SEQ ID NO: 1.

14. (canceled)

15. A nucleic acid comprising a nucleotide sequence that encodes a polypeptide according to claim 1.

16. A host or host cell comprising a nucleic acid according to claim 15.

17. A method for producing a polypeptide, said method comprising:
a. expressing, in a suitable host cell or host organism or in another suitable expression system, a nucleic acid according to claim 15; optionally followed by:
b. isolating and/or purifying the polypeptide.

18. A composition comprising at least one polypeptide according to claim 1, or a nucleic acid comprising a nucleotide sequence that encodes said polypeptide, optionally wherein the composition is a pharmaceutical composition which further comprises: at least one pharmaceutically acceptable carrier, diluent or excipient and/or adjuvant; and/or one or more further pharmaceutically active polypeptides and/or compounds.

19.-22. (canceled)

23. A method of treating acute myeloid leukemia (AML), wherein said method comprises administering, to a subject in need thereof, a pharmaceutically active amount of a polypeptide according to claim 1, a nucleic acid comprising a nucleotide sequence that encodes said polypeptide, or a composition comprising said polypeptide or said nucleic acid, optionally wherein the AML is relapsed and/or refractory AML.

24.-26. (canceled)

27. An immunoglobulin single variable domain (ISVD) capable of binding to CD33, wherein the ISVD comprises three complementarity determining regions (CDR1 to CDR3, respectively), wherein CDR1 to CDR3 correspond to the sequences of CDR 1 to CDR3 of the ISVD of SEQ ID NO: 3 or to sequences having 2 or 1 amino acid difference(s) with the CDR1 to CDR3 sequences of SEQ ID NO: 3; optionally:
a. in which:
i. CDR1 (Abm numbering) has the amino acid sequence of SEQ ID NO: 7 or has 2 or 1 amino acid difference(s) with SEQ ID NO: 7;
ii. CDR2 (Abm numbering) has the amino acid sequence of SEQ ID NO: 11 or has 2 or 1 amino acid difference(s) with SEQ ID NO: 11; and
iii. CDR3 (Abm numbering) has the amino acid sequence of SEQ ID NO: 15 or has 2 or 1 amino acid difference(s) with SEQ ID NO: 15; or
b. in which:
iv. CDR1 (Kabat numbering) has the amino acid sequence of SEQ ID NO: 35 or has 2 or 1 amino acid difference(s) with SEQ ID NO: 35;
v. CDR2 (Kabat numbering) has the amino acid sequence of SEQ ID NO: 39 or has 2 or 1 amino acid difference(s) with SEQ ID NO: 39; and
vi. CDR3 (Kabat numbering) has the amino acid sequence of SEQ ID NO: 43 or has 2 or 1 amino acid difference(s) with SEQ ID NO: 43.

28. An immunoglobulin single variable domain (ISVD) capable of binding to CD123, wherein the ISVD comprises three complementarity determining regions (CDR1 to CDR3, respectively), wherein CDR1 to CDR3 correspond to the sequences of CDR1 to CDR3 of the ISVD of SEQ ID NO: 4 or to sequences having 2 or 1 amino acid difference(s) with the CDR1 to CDR3 sequences of SEQ ID NO: 4; optionally:
a. in which:
i. CDR1 (Abm numbering) has the amino acid sequence of SEQ ID NO: 8 or has 2 or 1 amino acid difference(s) with SEQ ID NO: 8;
ii. CDR2 (Abm numbering) has the amino acid sequence of SEQ ID NO: 12 or has 2 or 1 amino acid difference(s) with SEQ ID NO: 12; and
iii. CDR3 (Abm numbering) has the amino acid sequence of SEQ ID NO: 16 or has 2 or 1 amino acid difference(s) with SEQ ID NO: 16; or
b. in which:
iv. CDR1 (Kabat numbering) has the amino acid sequence of SEQ ID NO: 36 or has 2 or 1 amino acid difference(s) with SEQ ID NO: 36;
v. CDR2 (Kabat numbering) has the amino acid sequence of SEQ ID NO: 40 or has 2 or 1 amino acid difference(s) with SEQ ID NO: 40; and
vi. CDR3 (Kabat numbering) has the amino acid sequence of SEQ ID NO: 44 or has 2 or 1 amino acid difference(s) with SEQ ID NO: 44.
